(19) United States
(12) Patent Application Publication
(10) Pub. No.: US 2005/0215901 A1
(43) Pub. Date: Sep. 29, 2005

(54) Interface for use between medical instrumentation and a patient

(76) Inventors: Thomas L. Anderson, Redmond, WA (US); Nathan J. Dale, Everett, WA (US); Peter G. Edelman, Mukilteo, WA (US); John M. Stiggelbout, Sausalito, CA (US); David M. Perozek, Mercer Island, WA (US); Lee Weng, Bellevue, WA (US); Jimin Zhang, Bellevue, WA (US); Robert Hubler, Woodinville, WA (US); Paul C. Leonard, Woodinville, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/038,351

(22) Filed: Jan. 18, 2005

Related U.S. Application Data
(60) Provisional application No. 60/537,034, filed on Jan. 20, 2004.

(51) Int. Cl. A61B 8/00; A61B 8/12

(52) U.S. Cl. 600/445

(57) ABSTRACT

Disclosed herein are methods, devices, compositions, and systems for providing an interface between medical instrumentation and a patient. In various embodiments, the interface provides a sterile barrier, acoustic coupler, and thermal insulator between the patient and a medical instrument. In some embodiments, an acoustic coupler interface is used between an ultrasound instrument and a patient. In some embodiments, the acoustic coupler comprises a thermoplastic elastomer (“TPE”) and in particular oil-enhanced or gelatinous TPEs that can be used in diagnostic and therapeutic (HIFU) ultrasound procedures.

### RELATED APPLICATION

This application claims priority from U.S. Provisional Application No. 60/537,034, filed on Jan. 20, 2004, which is incorporated herein by reference in its entirety.

### BACKGROUND OF THE INVENTION

1. Field of the Invention

The present invention relates generally to medical devices and methods. More specifically, the present invention relates to an interface for use between medical instrumentation and a patient. In one embodiment, the present invention relates to diagnostic and therapeutic ultrasound, and in particular, to various thermoplastic elastomers (“TPE's”) as acoustic transmission media.

2. Description of the Related Art

It is advantageous in many medical procedures to use a sterile barrier between the patient and medical instrumentation. Such sterile barriers may be necessary when the medical instrumentation cannot be easily sterilized. Furthermore, the use of disposable sterile barriers may be advantageous to reduce the time required for procedure preparation and to allow handling of the medical instrumentation by non-sterile personnel.

Elevated temperature treatments are used for a variety of purposes in medicine. In high intensity focused ultrasound (“HIFU”) treatments, ultrasonic energy is focused on a small spot within the body in order to heat tissues to a temperature sufficient to create a desired therapeutic effect. This technique can be used to selectively destroy unwanted tissue in the body by applying focused ultrasonic energy to a predetermined target area and sufficiently raising the native tissue temperatures to kill tissue without destroying the adjacent normal tissues. Other elevated-temperature treatments include selectively heating tissues to promote other physiological tissue changes or bio-effects—such as coagulation, collagen melting, or tissue adhesion—in a pre-determined volume of a patient's body. The specific physiological change that can be induced with HIFU will typically depend on a number of factors, including, but not limited to: the native tissue temperature; the composition of the tissue; and the characteristics of the ultrasonic energy being applied, such as frequency, intensity, beam focusing geometry, duty cycle, and duration of application.

HIFU heating is typically conducted using either discrete fixed-focus transducers (either single or multiple transducers), or using multiple ultrasonic transducers comprising an electronically controlled and driven array. For the case of the array, the individual array elements are actuated with a drive signal in order to emit therapeutic HIFU waves at a selected frequency and phase. Specific changes can be applied to the drive signals so that the therapeutic ultrasonic waves tend to constructively reinforce one another at a “focal location,” allowing the acoustic energies to be most intense in the volume of tissues located at the focal location. A significant advantage of HIFU as an energy delivery modality is its ability to deliver concentrated energy to a remote focal location with minimal or no lasting damage to intervening or adjacent tissues.

A drawback of using acoustic waves, whether for therapy or imaging, is that high frequency acoustic waves are reflected at gas-couplant media interfaces and, thus, do not travel efficiently in air. In order to efficiently propagate, or transmit, acoustic waves into a patient's body, the use of a transmission medium between the ultrasonic transducer and the patient's body is often needed. In some cases, a fluid gel is used to couple ultrasound energy between the ultrasound transducer and the patient's body. However, such a fluid gel does not provide a sterile barrier between the ultrasound transducer and the patient, a deficiency particularly important in sterile tissue field applications. Furthermore, in cases where the ultrasound energy is being applied to an open wound, use of a fluid gel may cause unwanted chemicals to enter the wound site and may not be effective when pressure is applied to the wound site.

Thus, there is a need for improved sterile barriers for use between medical instrumentation and the patient, particularly for use between ultrasound transducers and the patient.

### SUMMARY OF THE INVENTION

One aspect of the present invention is an ultrasound coupling pad, comprising a gelatinous thermoplastic elastomer mass adapted to permit transmission of ultrasound energy through the mass, the mass comprising a patient surface configured to transmit the acoustic energy to tissues of a patient either directly or through other materials and a transducer surface configured to receive the acoustic energy from one or more ultrasound transducers either directly or through other materials.

Another aspect of the present invention is an ultrasound coupling pad, comprising a gelatinous thermoplastic elastomer mass adapted to permit transmission of ultrasound energy through the mass and a housing contacting at least some surfaces of the mass for stably holding the mass, the housing adapted to couple to an ultrasound applicator, wherein when the housing is coupled to the ultrasound applicator, at least one surface of the mass is held in close proximity to one or more ultrasound transducers in the ultrasound applicator. In one embodiment, the housing comprises a tab or tab receptacle for coupling to the ultrasound applicator. In another embodiment, the housing comprises threads for coupling to the ultrasound applicator.

Another aspect of the present invention is an ultrasound coupling pad, comprising a gelatinous thermoplastic elastomer mass adapted to permit transmission of ultrasound energy through the mass and a housing contacting at least some surfaces of the mass for stably holding the mass, the housing comprising an adhesive coating on a least a portion of the housing's outer surface, the adhesive adapted to adhere the housing to a patient, wherein when the housing is adhered to the patient, at least one surface of the mass is held in close proximity to the patient. In one embodiment, the gelatinous thermoplastic elastomer mass can be removed from the housing while the housing is adhered to the patient.

Another aspect of the present invention is an ultrasound coupling pad, comprising a first gelatinous solid mass optimized to permit transmission of ultrasound energy having a first frequency through the mass and a second gelatinous solid mass comprising a different chemical composition than the first mass and optimized to permit transmission of ultrasound energy having a second frequency through the mass, wherein the second frequency is different from the first frequency. In one embodiment, the first gelatinous solid mass comprises a hydrogel and the second gelatinous solid mass comprises a thermoplastic elastomer. In one embodiment, the first gelatinous solid mass is optimized to permit transmission of ultrasound energy from an imaging ultrasound transducer and the second gelatinous solid mass is optimized to permit transmission of ultrasound energy from a therapeutic ultrasound transducer.

Another aspect of the present invention is a sterile barrier for use between a patient and an instrument, comprising a flexible sheath adapted to prevent passage of microbes from one side of the sheath to the other; the sheath comprising an openable seal, wherein when the seal is closed, the seal prevents passage of microbes from one side of the seal to the other; the sheath configured to have a predeployed state and a postdeployed state, wherein in the predeployed state, the seal is closed and the flexible sheath with closed seal form a continuous barrier having no opening therein and having no edges, the continuous barrier having an inside surface and an outside surface, wherein the inside surface is sterilized, and wherein in the postdeployed state, the flexible barrier is inverted such that the sterilized inside surface faces outward and the outside surface faces inward and is placed in contact with a medical instrument, thereby providing a barrier between the medical instrument and the sterilized surface of the sheath. In some embodiments, the seal comprises an adhesive. In some embodiments, the seal comprises Tyvek®. In some embodiments, the seal comprises a heat induced seal.

Another aspect of the present invention is a sterile barrier for use between a patient and an ultrasound applicator, comprising a flexible sheath adapted to prevent passage of microbes from one side of the sheath to the other, the sheath adapted to surround an ultrasound applicator and a gelatinous solid mass adapted to permit transmission of ultrasound energy through the mass and prevent passage of microbes from one side of the mass to the other, the mass coupled to the flexible sheath such that when the sheath surrounds the ultrasound applicator, the mass may be placed in close proximity to one or more ultrasound transducers in the ultrasound applicator. In some embodiments, the mass is coupled to the sheath by a housing that is coupled to the flexible sheath and contacts at least some surfaces of the gelatinous solid mass and is adapted to couple the gelatinous solid mass to the ultrasound applicator.

In some embodiments, the flexible sheaths described above comprise polyurethane, polyethylene, or other suitable polymers. In some embodiments, at least a portion of the flexible sheath comprises material that is more rigid than other portions of the flexible sheath. In some embodiments, the sheath further comprises one or more tabs attached to the outside surface. In some embodiments, at least one of the tabs comprises a bar code. In some embodiments, at least one of the tabs comprises a radio frequency identification (RFID) feature. In some embodiments, at least one of the tabs comprises a radio frequency surface acoustic wave (RFSAW) identification feature.

Another aspect of the present invention is an ultrasound coupling pad kit, comprising a sterilized gelatinous thermoplastic elastomer mass adapted to permit transmission of ultrasound energy through the mass and a protective barrier surrounding at least a portion of the mass, the barrier adapted to prevent passage of microbes from one side of the barrier to the other, thereby maintaining sterility of the mass, at least a portion of the barrier adapted to be removed from surrounding the mass prior to use of the mass for transmission of ultrasound energy.

Another aspect of the present invention is a kit for a sterile barrier for use between a patient and an ultrasound applicator, comprising a flexible sheath adapted to prevent passage of microbes from one side of the sheath to the other, the sheath adapted to surround an ultrasound applicator, at least one surface of the flexible sheath sterilized and a gelatinous solid mass adapted to permit transmission of ultrasound energy through the mass, the mass comprising a sterilized patient surface configured to transmit the acoustic energy to tissues of a patient either directly or through other materials and a transducer surface configured to receive the acoustic energy from one or more ultrasound transducers in the ultrasound applicator either directly or through other materials. In one embodiment, the gelatinous solid mass is coupled to the flexible sheath.

Another aspect of the present invention is an ultrasound coupling pad, comprising a gelatinous thermoplastic elastomer mass adapted to permit transmission of ultrasound energy through the mass and a means for coupling the mass to an ultrasound applicator.

Another aspect of the present invention is an ultrasound coupling pad, comprising a gelatinous thermoplastic elastomer mass adapted to permit transmission of ultrasound energy through the mass and a means for coupling the mass to a patient.

Another aspect of the present invention is a sterile barrier for use between a patient and an ultrasound applicator, comprising a gelatinous solid mass adapted to permit transmission of ultrasound energy through the mass and prevent passage of microbes from one side of the mass to the other, a means for preventing passage of microbes from at a least a portion of an ultrasound applicator's surface to a patient, and a means for coupling the gelatinous solid mass to the means for preventing passage of microbes.

Another aspect of the present invention is a method of transmitting ultrasound energy from an ultrasound transducer to tissue of a patient, comprising positioning one surface of a gelatinous thermoplastic elastomer mass in close proximity to an ultrasound transducer, the mass adapted to permit transmission of ultrasound energy through the mass; positioning another surface of the gelatinous thermoplastic elastomer mass in close proximity to tissue of a patient; and energizing the ultrasound transducer such that ultrasound energy passes from the ultrasound transducer, through the mass, and into the tissue of the patient. In one embodiment, the step of positioning a surface of the mass in close proximity to an ultrasound transducer is performed prior to the step of positioning a surface of the mass in close proximity to tissue of a patient. In another embodiment, the step of positioning a surface of the mass in close proximity to tissue of a patient is performed prior to the step of positioning a surface of the mass in close proximity to an ultrasound transducer.

Another aspect of the present invention is a method of acoustic hemostasis, comprising positioning one surface of a gelatinous solid mass in close proximity to a wound on a patient, the mass adapted to permit transmission of ultrasound energy through the mass; applying sufficient pressure to the gelatinous solid mass so as to temporarily stop or slow bleeding from the wound; and transmitting ultrasound energy through the mass into the wound, thereby stopping bleeding from the wound. In one embodiment, the step of positioning a surface of the mass in close proximity to a wound on a patient comprises directly contacting the wound with the mass. In another embodiment, the step of positioning a surface of the mass in close proximity to a wound on a patient comprises applying an acoustic gel or liquid between the mass and the wound. In one embodiment, the step of positioning a surface of the mass in close proximity to a wound on a patient comprises coupling the mass to the patient. In one embodiment, the step of applying pressure to the mass comprises contacting the mass with an ultrasound applicator and applying force to the ultrasound applicator.

Another aspect of the present invention is a method of providing a sterile barrier between a patient and a medical instrument, comprising opening a seal in a flexible sheath, the sheath adapted to prevent passage of microbes from one side of the sheath to the other, the seal disposed on the sheath, wherein when the seal is closed, the seal prevents passage of microbes from one side of the seal to the other, wherein prior to opening the seal, the flexible sheath with closed seal forms a continuous barrier having no opening therein and having no edges, the continuous barrier having an inside surface and an outside surface, wherein the inside surface is sterilized, contacting a medical instrument with the outside surface of the flexible sheath; and inverting the flexible sheath so that the sterilized inside surface faces outward and the outside surface faces inward in contact with the medical instrument, thereby providing a barrier between the medical instrument and the sterilized surface of the sheath. In one embodiment, the opening of the seal and inverting of the flexible sheath is accomplished by pulling on tabs fixed on the outside surface. In one embodiment, the step of opening of the seal is performed prior to the step of contacting a medical instrument with the outside surface. In one embodiment, the step of contacting a medical instrument with the outside surface is performed prior to the step of opening of the seal. In one embodiment, the opening, contacting, and inverting steps are performed by non- sterile personnel. In one embodiment, the contacting step is performed by partially inverting the flexible sheath prior to the opening step.

Another aspect of the present invention is a method of providing a sterile barrier for use between a patient and an ultrasound applicator, comprising inserting an ultrasound applicator within a flexible sheath adapted to prevent passage of microbes from one side of the sheath to the other and coupling a gelatinous solid mass to the ultrasound applicator in close proximity to one or more ultrasound transducers, the gelatinous solid mass adapted to permit transmission of ultrasound energy through the mass and prevent passage of microbes from one side of the mass to the other. In one embodiment, the gelatinous solid mass is coupled to the flexible sheath such that the coupling step is performed after the ultrasound applicator is at least partially inserted within the flexible sheath. In one embodiment, the coupling step comprises sandwiching the flexible sheath between the ultrasound applicator and the gelatinous solid mass. In one embodiment, prior to the coupling step, an acoustic gel or liquid is applied to the gelatinous solid mass so that the gel or liquid is disposed between the solid mass and the one or more ultrasound transducers after performing the coupling step.

Another aspect of the present invention is a method of providing a sterile barrier for use between a patient and an ultrasound applicator, comprising coupling a gelatinous solid mass to an ultrasound applicator in close proximity to one or more ultrasound transducers, the gelatinous solid mass adapted to permit transmission of ultrasound energy through the mass and prevent passage of microbes from one side of the mass to the other, the mass comprising a sterilized patient surface and a protective barrier applied to the surface, whereby sterility of the surface is maintained and removing the protective barrier from the patient surface. In one embodiment, the removing step is performed after the coupling step.

Another aspect of the present invention is a method of providing a sterile barrier for use between a patient and an ultrasound applicator, comprising removing a protective barrier from a patient surface of gelatinous solid mass, the mass adapted to permit transmission of ultrasound energy therethrough and prevent passage of microbes from one side of the mass to the other, the protective barrier applied to the patient surface of the mass to maintain sterility of the surface prior to use; and coupling the gelatinous solid mass to a patient so that the patient surface is in close proximity to the patient.

In some embodiments, the protective barriers described above comprise a film and the removing steps comprise peeling off the film from the patient surface. In one embodiment, the film comprises a polymer such as polyurethane, Teflon, mylar, polyethylene terephthalate or polyethylene. In one embodiment, the protective barrier also covers an adhesive on a housing coupled to the mass prior to removal of the barrier.

Another aspect of the present invention is a method of optimizing a thermoplastic elastomer for use as an acoustic coupler, the thermoplastic elastomer comprising a soft block segment, a hard block segment, and at least one modifier, the modifier including a soft block compatible modifier or a hard block compatible modifier, the method comprising varying at least one of the soft block segment, the hard block segment, and the modifieruntil an elastomer having one or more desired properties is obtained. In one embodiment, the varying comprises varying the relative amounts of the soft block segment, the hard block segment, and the modifier. In another embodiment, the varying comprises varying the composition of at least one of the soft block segment, the hard block segment, and the modifier. In another embodiment, at least one desired property is an acoustic property. In another embodiment, the acoustic property comprises acoustic impedance. In another embodiment, the acoustic property comprises acoustic attenuation. In another embodiment, at least one desired property is a mechanical property. In another embodiment, the mechanical property comprises compression force transmission. In another embodiment, the mechanical property comprises elasticity. In another embodiment, at least one desired property is a thermal insulative property.

In some embodiments, the gelatinous solid masses set forth in any of the aspects described above comprise a hydrogel. In other embodiments, the gelatinous solid masses comprise a gelatinous thermoplastic elastomer. In some embodiments, the gelatinous thermoplastic elastomer mass comprises a plurality of thermoplastic elastomers having different compositions. In one embodiment, the gelatinous thermoplastic elastomer mass comprises styrene. In some embodiments, the gelatinous thermoplastic elastomer mass comprises one or more soft block segments selected from the group consisting of butadiene, isoprene, isoprene-butadiene, ethylene-butylene, ethylene-propylene, ethylene-butylene-ethylene-propylene, and ethylene-ethylene-propylene. In some embodiments, the gelatinous thermoplastic elastomer mass comprises an oil.

In some embodiments, the gelatinous solid masses, either hydrogel or thermoplastic elastomer, directly contacts a patient. In other embodiments, an acoustic gel or liquid is disposed between the patient and the gelatinous solid masses. In some embodiments, the gelatinous solid masses directly contact an ultrasound transducer. In other embodiments, an acoustic gel or liquid is disposed between the transducer and the gelatinous solid masses. In some embodiments, the patient surface of the gelatinous solid masses are convex shaped. In some embodiments, a reservoir is provided that contains an acoustic gel or liquid, wherein the reservoir is adapted to dispense the acoustic gel or liquid onto the patient and/or transducer surfaces.

In some embodiments, a removable protective barrier is disposed on the patient surface of the gelatinous solid masses described above, either hydrogel or thermoplastic. In some embodiments, an acoustic gel or liquid is disposed between the protective barrier and the gelatinous solid masses

In some embodiments, the gelatinous solid masses described above, either hydrogel or thermoplastic elastomer, are coupled to an ultrasound transducer. In some embodiments, the masses are coupled to a patient.

### BRIEF DESCRIPTION OF THE DRAWINGS

FIG. 1 illustrates an ultrasound transducer apparatus with an acoustic couplant.

FIG. 2 illustrates a solid acoustic couplant material in a dimensionally stable flat sheet.

FIGS. 3A-3C illustrate various conformal ultrasound transducer acoustic couplant covers and acoustic couplant block configurations.

FIG. 4 illustrates an ultrasound applicator with an acoustic couplant pad assembly that can be attached to the ultrasound applicator.

FIG. 5 is a cut away view of an acoustic couplant pad assembly and a couplant applicator assembly for use in attaching the acoustic couplant pad assembly to an ultrasound applicator.

FIGS. 6A-6E illustrate various acoustic couplant pad assembly configurations.

FIG. 7 illustrates an acoustic couplant pad assembly that can be adhered to the surface of a patient.

FIG. 8 illustrates an acoustic couplant pad assembly that can be adhered to the surface of a patient and from which a section of the acoustic couplant material can be temporarily lifted off of the surface of the patient.

FIG. 9 illustrates another embodiment of the acoustic couplant pad assembly of FIG. 7.

FIG. 10A-10B illustrate a plan view of an acoustic couplant pad assembly containing two different acoustic couplant materials.

FIGS. 11A-11G illustrate cross-sectional views of various acoustic couplant pad assemblies containing multiple materials.

FIGS. 12A-12C illustrate the general chemical composition of thermoplastic elastomers.

FIGS. 13A-13E illustrate cross-sectional views of various gel or liquid based acoustic couplant pads.

FIG. 14 illustrates a pre-sterilized sterile barrier that can be deployed over a medical instrument.

FIG. 15 illustrates another configuration of the sterile barrier of FIG. 14.

FIGS. 16A-16D illustrate the deployment of a pre-sterilized sterile barrier over an ultrasound applicator.

FIG. 17 illustrates a cross-sectional view of a sterile barrier for use in isolating a catheterization site from an ultrasound applicator.

### DETAILED DESCRIPTION OF THE EMBODIMENTS

In some embodiments of the present invention, a patient interface is provided for use between medical instrumentation and a patient. The medical instrumentation for use with the patient interfaces disclosed herein may be employed both on the surface of a patient as well as within a patient's body, such as within a patient's cavity. In various embodiments, the patient interface may be coupled to the medical instrument itself, adhered to the surface of the patient, or freely placed between the medical instrument and the patient. In some embodiments, the patient interface provides a sterile barrier between the medical instrument and the patient. In some embodiments, the patient interface may incorporate additional functionality such as providing acoustic coupling between an ultrasound transducer and tissue of a patient or providing a thermal barrier between medical instrumentation and the patient. In some embodiments, the patient interface may be provided as a single use, disposable article. In such embodiments, the patient interface may advantageously be presterilized and packaged so as to maintain sterility until it is ready for use.

Solid Acoustic Coupling Interfaces

In one embodiment, a sterile barrier is provided between an ultrasound transducer and the patient. In one embodiment, the sterile barrier is adapted to permit the transmission of ultrasound energy through the barrier. In one embodiment, the sterile barrier is a dimensionally stable solid so as to permit compression of the sterile barrier against the patient without the barrier substantially losing its shape. As used herein, a “dimensionally stable solid” refers to a material that upon removal of a compressive or stretching force returns substantially to the same shape.

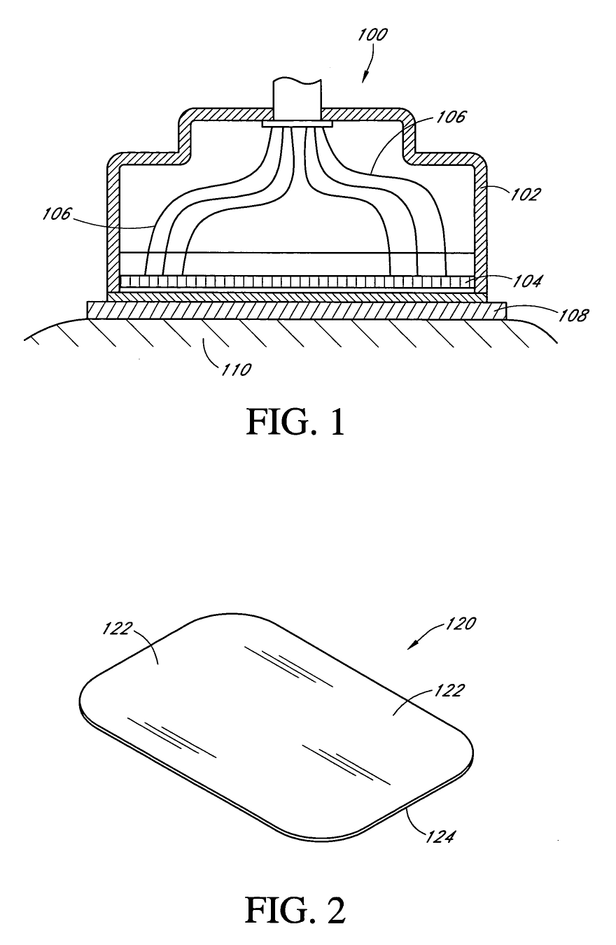

FIG. 1 generically illustrates an ultrasound applicator in combination with a dimensionally stable solid acoustic coupler. Ultrasound applicator 100 comprises a housing 102 that contains an array of transducer elements 104. The transducer elements 104 transmit diagnostic interrogation and/or therapeutic acoustic waves in response to electrical signals supplied through an interconnecting means (such as a cable and lead wires 106) from a control unit. A dimensionally stable solid acoustic coupler medium 108 is provided between the ultrasound applicator 100 and tissue 110 of a patient. In one advantageous embodiment, the coupler medium 108 is adapted to transmit ultrasonic waves efficiently from the ultrasound transducers 104 to the tissue 110 of the patient with little or no deterioration of the propagated waves. The acoustic properties of the transmission media may advantageously be similar to that of the tissues below; specifically acoustic impedance and velocity may be matched. Moreover, particularly in therapeutic applications, the acoustic attenuation coefficient of the media may be low, or alternatively, optimized for a particular application or treatment. Further, the physical properties (e.g., elasticity, hardness, adhesion, lubricity, thermal conductivity, etc.) of the media can be appropriate for its intended use (e.g., whether the media is for use during a diagnostic or therapeutic procedure or for use with a transducer configured to emit ultrasound waves having a particular frequency, energy, etc); and finally, deterioration of the media under harsh operational conditions (such as high heat, manual manipulation, long procedure times, etc.) may advantageously be minimized, especially if the media is to be employed for therapeutic applications. In one embodiment, the coupler medium 108 is also adapted to insulate the tissue 110 from heat conducted from transducer elements 104. Thus, tissue deeper within the patient can be selectively heated by ultrasonic energy while tissue near the surface of the patient 110 receives little heating.

In one embodiment, a method of transmitting ultrasound energy from an ultrasound transducer into tissue of a patient is provided. A solid acoustic couplant material is positioned between the ultrasound transducer and the tissue of the patient. One surface of the solid acoustic couplant material is placed in close proximity to the tissue of the patient. Another surface of the acoustic couplant material is placed in close proximity to the ultrasound transducer. The ultrasound transducer is then energized to provide ultrasound energy through the solid acoustic couplant material and into the tissue of the patient.

FIG. 2 illustrates an embodiment wherein a flat acoustic couplant sheet 120 is provided. The couplant sheet 120 comprises a transducer contacting surface 122 and a patient or tissue contacting surface 124. In one embodiment, the acoustic couplant sheet 120 is non-adhesive, non-sticky, transparent allowing a user a view of the tissues or patient below the couplant sheet 120, non-toxic, and dimensionally stable. Thus, the couplant sheet 120 may be placed on the surface of the patient so that the patient contacting surface 124 contacts the patient. The patient surface can be configured to contact a patient's skin, to contact other tissue, or for intracorporeal use. An ultrasound applicator may be positioned on the transducer contacting surface 122 prior to application of ultrasonic energy. The couplant sheet 120 can be readily configured to be of any size, dimension and shape and can be optimized for a specific intended use. In this embodiment, a dimensionally stable, solid acoustic coupler is provided that is not adhered to either the patient or the ultrasound applicator and thus may be easily moved from one location to another on the patient or may be employed with multiple ultrasound applicators during the same procedure. If desired, acoustic coupling between the ultrasound applicator and the transducer contacting surface 122 may be enhanced by the application of an acoustic gel or liquid between the ultrasound transducers and the transducer contacting surface 122. Similarly, acoustic gel or liquid may be placed between the patient contacting surface 124 and the patient to enhance acoustic coupling from the couplant sheet 120 to tissues of the patient.

In some embodiments solid acoustic couplers such as acoustic couplant sheet 120 may comprise removable protective sheets to prevent the acoustic couplant sheet 120 from drying out and to maintain sterility of the sheet 120. Thus for example, transducer contacting surface 122 and patient contacting surface 124 may both contain a protective sheet that may be peeled away prior to use. Any suitable sterile sheet may be used for the protective sheet such as a polymer (e.g., polyurethane, Teflon, mylar, polyethylene, PET etc.). Alternatively, acoustic couplant sheet 120 may be provided in a sealed package from which the pre-sterilize sheet 120 may be removed prior to use.

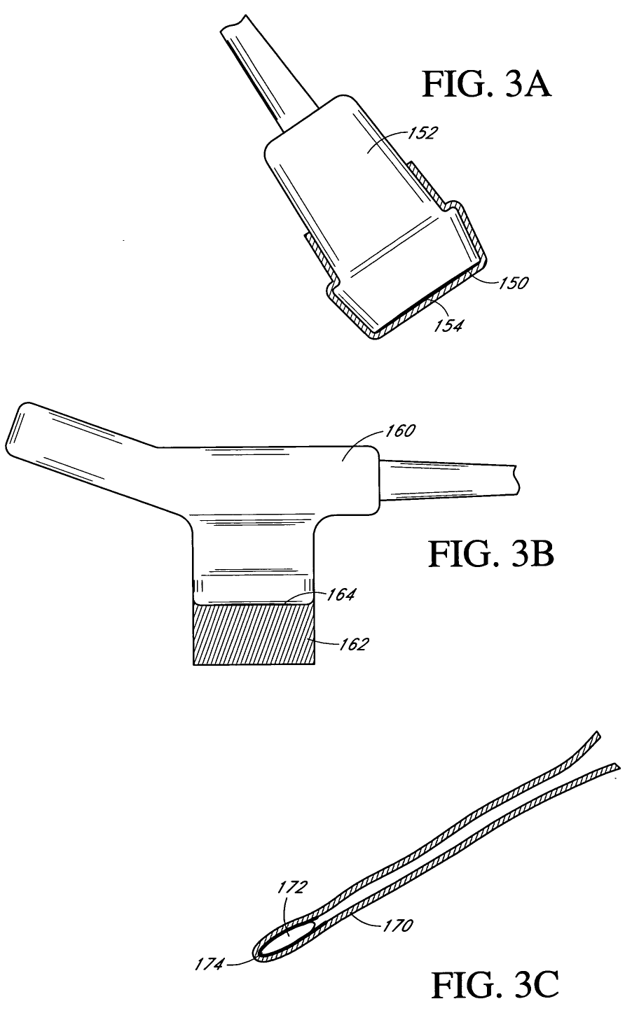

FIGS. 3A through 3C depict various embodiments where a dimensionally stable solid acoustic coupler is coupled directly to an ultrasound applicator. In these embodiments, the dimensionally stable solid acoustic couplers are conformal with at least a portion of an ultrasound applicator. In FIG. 3A, an acoustic couplant sheet 150 is conformal with the end an ultrasound applicator 152 that contains an ultrasound transducer 154. The acoustic couplant sheet 150 may also cover other portions of the ultrasound applicator 152 in order to provide a sterile barrier between the ultrasound applicator 152 and the patient. The acoustic couplant sheet 150 can be relatively thin. In one embodiment, sheet 150 is between about 0.02-10 mm thick. In one embodiment, sheet 150 is between about 1 mm and 5 mm thick. Optionally, the various commercially available or otherwise known ultrasound gels, liquids, and the like may be disposed between the transducer 154 and the sheet 150 to eliminate or minimize the presence of air or air bubbles trapped between the transducer 154 and sheet 150 that can decrease or impair acoustic transmission and efficient acoustic transfer. Preferably, the gel or liquid used in conjunction with the present invention is non-toxic and bio-compatible.

In FIG. 3B, ultrasound applicator 160 comprises a block 162 of dimensionally stable solid acoustic couplant material disposed over ultrasound transducer 164. In this embodiment, the acoustic couplant material 162 covers only the ultrasound transducer 164, however, an additional sterile barrier may be provided around the rest of ultrasound applicator 160 to maintain sterility between it and the patient.

In FIG. 3C, an ultrasound couplant sheet 170 is provided that completely surrounds ultrasound applicator 172. The sheath 170 is advantageously conformal with the ultrasound transducer 174 of the ultrasound applicator 172. The sheath 170 thus provides a continuous microbial barrier between the ultrasound applicator 172 and the patient and may be advantageously used in intracavity transducers.

In embodiments where the solid acoustic couplant material is coupled to the ultrasound applicator, it may be coupled by any suitable means known in the art. Non-limiting examples include use of adhesives and structures such as snap features or threads that hold the couplant material to the ultrasound applicators. In some advantageous embodiments, the solid acoustic couplant material is coupled to the ultrasound applicator such that at least one surface of it is in close proximity to one or more ultrasound transducers in the ultrasound applicator. By “close proximity,” it is meant that the solid acoustic couplant material is close enough to the ultrasound transducers so that ultrasonic energy may be transmitted from the transducers into the couplant material, either directly or through thin layers of other material such as acoustic gel or liquid or a thin film.

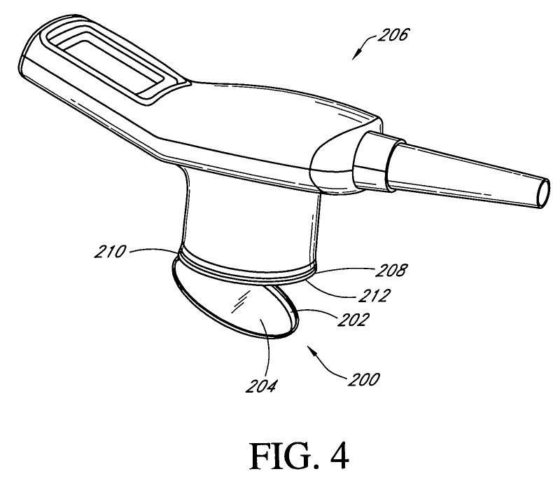

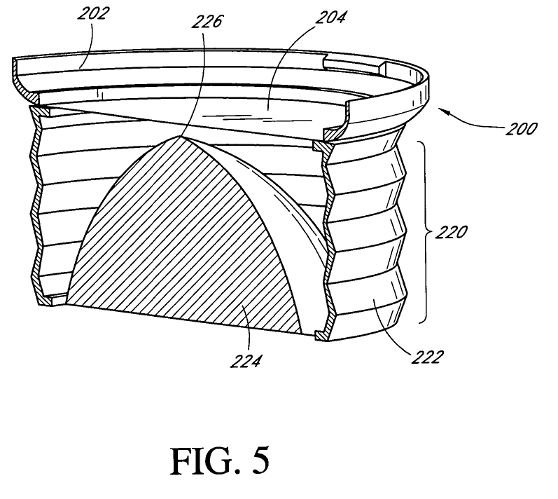

FIG. 4 depicts another embodiment where solid acoustic couplant material is coupled to an ultrasound applicator. In FIG. 4, the solid acoustic couplant material is contained within a couplant pad assembly 200. The couplant pad assembly 200 comprises a housing 202 that serves to provide structural support to solid acoustic couplant material 204 and to couple the couplant pad assembly to the ultrasound applicator 206. The ultrasound applicator 206 comprises a receiving structure 208 for engaging housing 202. Any suitable structures may be used for coupling housing 202 and receiving structure 208. Non-limiting examples include snap features and threads. In one embodiment, housing 202 snaps to receiving structure 208 via a plurality of snap tabs. Advantageously, one snap tab at location 210 may be engaged and then couplant pad assembly 200 tilted around engagement point 210 until additional snap features are engaged. In this manner, the acoustic couplant material 204 may be placed in contact with ultrasound transducer surface 212 from one end of the acoustic couplant material 204 to the other. Thus, any air bubbles may be squeezed out as the acoustic couplant pad assembly 200 is attached to the ultrasound applicator 206. Acoustic couplant pad assembly 200 can advantageously provide a sterile barrier between ultrasound transducers 212 and a patient as well as providing acoustic coupling between the transducers 212 and the patient. In some embodiments, a sheet of material may be provided on the surfaces of acoustic couplant pad assembly 200 to maintain sterility of the surfaces prior to use. In some embodiments, acoustic coupling gel or liquid may be provided between the surfaces of the ultrasound transducer 212 and the acoustic couplant material 204 to further ensure that air bubbles are excluded between the surfaces. Similarly, acoustic coupling gel or liquid may be disposed between the patient and the acoustic couplant material 204. In various embodiments, the acoustic couplant material 204 may be homogenous or may comprise a plurality of materials as will be described in further detail below.

In one embodiment, the surface of acoustic couplant material 204 that faces ultrasound transducers 212 may be convex shaped. In this embodiment, the acoustic couplant pad assembly 200 may be attached to the ultrasound applicator 206 by connecting it straight onto the ultrasound applicator 206 rather than tilting it as depicted in FIG. 4. Because the surface of the acoustic couplant material 204 is convex, any air bubbles may be squeezed to the sides as the surface of the acoustic couplant material 204 is pressed onto the ultrasound transducers 212.

FIG. 5 depicts another embodiment of an acoustic couplant pad assembly 200 in conjunction with a couplant applicator assembly 220. The couplant applicator assembly 220 facilitates connecting acoustic couplant pad assembly 200 to an ultrasound applicator without air bubbles being trapped between the acoustic couplant material 204 and the ultrasound transducers. Couplant applicator assembly 220 may comprise collapsible sides 222 and center projection 224. Collapsible sides 222 may advantageously be formed of a flexible material, such as a rubber-like material. In one embodiment, collapsible sides 222 may be shaped to facilitate collapsing, such as by having an accordion shape. Those of skill in the art will appreciate multiple materials and shapes that may be used to provide collapsible functionality. In one embodiment, center projection 224 may advantageously be formed of a compressible material, such as a foam material. Those of skill in the art will appreciate numerous compressible materials that may be used.

Prior to attaching acoustic couplant pad assembly 200 to an ultrasound applicator, the acoustic couplant pad assembly 200 may be removably attached to collapsible sides 222. In one embodiment, center projection 224 does not contact acoustic couplant material 204 when collapsible sides 222 are not collapsed. The acoustic couplant pad assembly may be coupled with the ultrasound applicator by pressing the applicator against housing 202. The pressure applied by the ultrasound applicator causes collapsible sides 222 to partially collapse. This collapse moves the acoustic couplant material 204 into contact with the tip 226 of the center projection 224. Center projection 224 forces acoustic couplant material 204 to deform with the center of the material 204 being elevated. The raised center of acoustic couplant material 204 contacts the ultrasound transducers. As additional pressure is applied by the ultrasound applicator, the center projection 224 forces more of the acoustic couplant material 204 to contact the ultrasound transducers. The shape and compressibility of the center projection 224 may be selected so that continuing pressure applied by the ultrasound applicator causes the acoustic couplant material 204 to gradually contact the ultrasound transducers from the center of the material 204 moving towards the periphery. This action forces any air bubbles out the sides, thus ensuring an air bubble-free interface between the acoustic couplant material 204 and the ultrasound transducers. After enough force is applied by the ultrasound applicator, the housing 202 will coupled to the ultrasound applicator. After coupling, the couplant applicator assembly 220 may be removed from the acoustic couplant pad assembly 200. Center projection 224 may have any suitable convex shape such as a dome, cone, or pyramid shape.

In one embodiment, the acoustic applicator assembly 220 and acoustic couplant pad assembly 200 may be pre-packaged in a removably coupled state. In one embodiment, the acoustic applicator assembly 220 provides sterile protection of the patient side of the acoustic couplant material 204 prior to use.

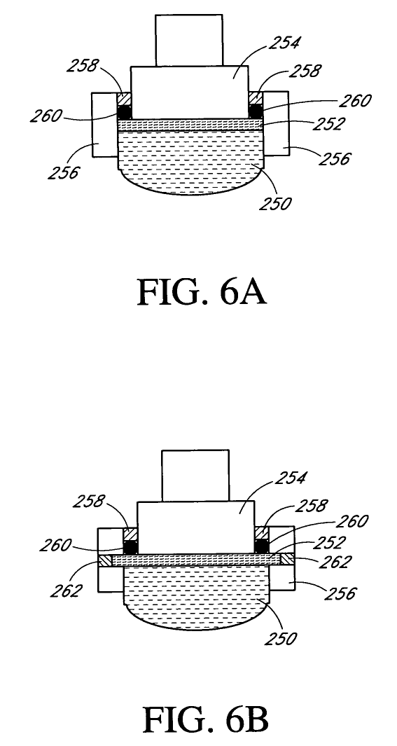

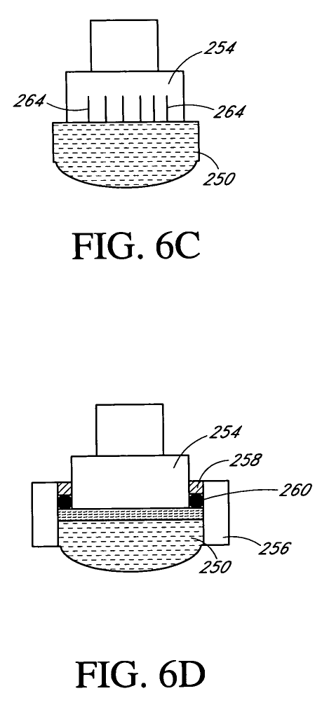

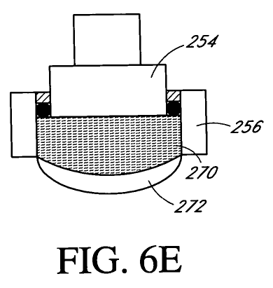

FIGS. 6A through 6E depict various embodiments of acoustic couplant pad assemblies engaged with an ultrasound applicator. In FIG. 6A, the acoustic couplant material comprises both solid acoustic couplant material 250 and an acoustic gel or liquid 252. The acoustic gel or liquid 252, helps prevent air bubbles from forming between the ultrasound transducer 254 and the solid acoustic couplant material 250. The acoustic couplant pad assembly comprises housing 256 for containing the acoustic couplant materials 250 and 252 and for engaging with connection features 258 on the ultrasound applicator. Connection features 258 on the ultrasound applicator provide a secure connection between the acoustic couplant pad assembly and the applicator and provides enough vertical force on the acoustic couplant pad assembly so that the acoustic couplant materials 250 and 252 are forced against the ultrasound applicator 254. Connection features 258 may be any suitable structures. Non-limiting examples include clips or threads. The acoustic couplant pad assembly may also comprise an O-ring 260 or other suitable seal. The O-ring 260 creates a seal between the acoustic couplant pad assembly and the ultrasound applicator. The seal prevents the acoustic gel or liquid 252 from leaking out of the acoustic couplant pad assembly and maintains a sterile seal between the ultrasound applicator and the acoustic couplant pad assembly.

FIG. 6B depicts a similar embodiment. Again, a solid acoustic couplant material 250 and an acoustic liquid or gel 252 is provided. The acoustic couplant materials are contained within housing 256, which may be attached to ultrasound applicator 254 using the attachment features 258. O-rings 260 provide a seal between the acoustic couplant pad assembly and the ultrasound applicator. In addition, one way vents 262 may be provided that allow for removal of air from the acoustic gel or liquid 252 and from the interfaces between the acoustic gel or liquid 252 and the ultrasound transducer 254 and solid acoustic couplant material 250. Removal of air through the one-way vents 262 may be accomplished by providing pressure on the solid acoustic couplant material 250. Alternatively, suction may be provided to one-way vents 262 to remove the air.

FIG. 6C depicts an embodiment where housing 256, attachment features 258, O-ring 260, and acoustic gel or liquid 252 are not necessary. A solid acoustic couplant material 250 is coupled to ultrasound transducer 254 by applying vacuum to vacuum vents 264. The suction provided by vacuum vents 264 forces the solid acoustic couplant material 250 onto the ultrasound transducer 254 with sufficient force that any air bubbles are eliminated between the ultrasound transducer 254 and the acoustic solid couplant material 250. Thus, the acoustic gel or liquid is not required. Furthermore, the suction provided by vents 264 provides a seal between the solid acoustic couplant material 250 and the ultrasound transducer 254 such that O-rings 260 are not needed.

FIG. 6D depicts another embodiment where acoustic gel or liquid 260 is not required. In this embodiment, housing 256, connection features 258, and O-rings 260 are present, however, the solid acoustic couplant material 250 contains sufficient properties that natural wicking occurs between the solid acoustic couplant material 250 and the ultrasound transducer 254. The natural wicking properties of the solid acoustic couplant material 250 may be due to a liquid content, such as water or oil, within the solid acoustic couplant material 250, so that liquid moves out of the acoustic couplant material 250 and displaces any air that may exist at the interface with the ultrasound transducer 254.

FIG. 6E depicts another embodiment where a single acoustic couplant material 270 is utilized. Material 270 directly contacts ultrasound transducer 254. The acoustic couplant material 270 may have natural wicking properties or may consist of acoustic gel or liquid so that efficient acoustic coupling is obtained between the material 270 and the ultrasound transducer 254. A thin shell material 272 may be provided on the patient side of the acoustic couplant material 270. This material serves the function of holding the acoustic couplant material 270 within housing 256 as well as preventing the acoustic couplant material 270 from drying out. Non-limiting examples of a suitable thin shell material 272 are polyurethane, polyethylene, or other suitable polymers. The thin shell material 272 is not necessarily a good acoustic coupler. However, because the thin shell material 272 is thin, the bulk acoustic coupling properties of the material are not as important.

The acoustic couplant pad assemblies depicted in FIGS. 6A through 6B include acoustic couplant materials having a convex shape on the patient interfacing surface. Such a convex shape provides several advantages. In one embodiment, the acoustic couplant pad assemblies are used with an ultrasound applicator to effect acoustic hemostasis following a catheterization procedure. The convex shape of the acoustic couplant materials allows the user to apply a concentrated force in a small area through the acoustic couplant assembly, thereby effecting temporary stoppage of blood flow from the catheter wound site during the acoustic hemostasis procedure. The convex shape of the acoustic couplant materials also promotes the exclusion of air bubbles between the acoustic couplant material and the patient. As the acoustic couplant material is pressed against the patient, the convex shape of the material forces air bubbles to the sides. In other embodiments, acoustic couplant materials having a flat patient surface are used.

Acoustic couplant material that can be coupled with ultrasound applicators as described above may be integral with the applicators, or alternatively, configured to be removable and/or disposable.

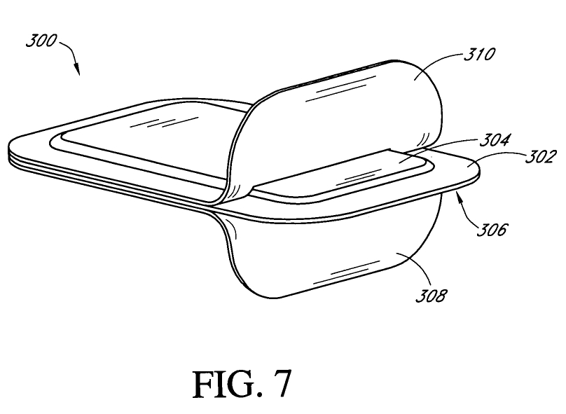

FIG. 7 depicts an embodiment of an acoustic couplant pad assembly 300 that can be secured to the surface of a patient prior to use in an ultrasound procedure. The acoustic couplant pad assembly 300 comprises a housing 302 that has the shape of a frame. The housing 302 holds a solid acoustic couplant material 304. The patient side of housing 302 may comprise an adhesive 306 that allows the housing 302 to be secured to the skin of a patient. Housing 302 may also comprise labels and/or markings that identify the acoustic couplant pad assembly 300, provide usage information, and/or provide guides to aid in positioning the acoustic couplant pad assembly 300 and an acoustic applicator to be used with the assembly 300. A film 308 may be disposed on the patient surface of the acoustic couplant pad assembly 300 to maintain the sterility of the solid acoustic couplant material 304, to protect it from drying out, and to cover the adhesive surface 306 prior to use. A film 310 may be disposed on the transducer surface of the acoustic couplant pad assembly 310 to similarly protect the acoustic couplant pad material 304 from drying out and to maintain sterility. Prior to use, medical personnel may remove films 308 and 310 and secure the acoustic couplant material to the surface of a patient using the adhesive surface 306. An ultrasound applicator may then be placed on the top surface of the acoustic couplant material 304 and the procedure commenced. Optionally, an acoustic gel or liquid media is disposed on the top and/or bottom surfaces of the solid acoustic couplant material 304. The acoustic gel or liquid media may be disposed between solid acoustic couplant material 304 and films 308 and 310 so that the acoustic couplant pad assembly 300 is ready for use as soon as films 308 and 310 are removed. Alternatively, the acoustic gel or liquid may be applied after removal of films 308 and 310. The acoustic couplant pad assembly 300 can be configured to have any number of different shapes, sizes, and dimensions; however, these properties would be determined by the intended acoustic application and the anatomical structure and location (e.g., dermal, intracavity) of the tissues or area to be treated.

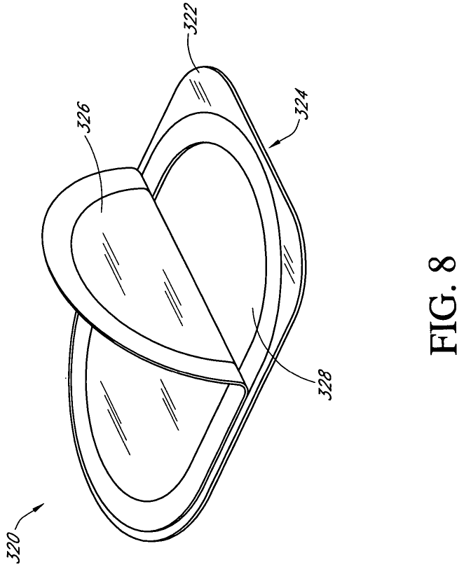

FIG. 8 depicts another embodiment of an acoustic couplant pad assembly 320 that may be secured to the surface of the patient. The acoustic couplant pad assembly 320 comprises a housing 322. The bottom patient surface of the housing 322 may comprise an adhesive 324 for securing the acoustic couplant pad assembly 320 to a patient. Housing 322 may also comprise labels and/or markings that identify the acoustic couplant pad assembly 320, provide usage information, and/or provide guides to aid in positioning the acoustic couplant pad assembly 320 and an acoustic applicator to be used with the assembly 320. The acoustic couplant pad assembly 320 also comprises a solid acoustic couplant material 326 for coupling ultrasound energy between an ultrasound applicator and the patient. In this embodiment, a section of the solid acoustic couplant material 326 may be temporarily lifted off of the patient surface and the housing 322, thereby leaving an opening 328 on the patient surface normally covered by the couplant material 326. Catheters and other objects may be placed through the opening 328 during deployment of the acoustic couplant pad assembly 320. After accessing the patient through opening 328, the couplant material 326 may be replaced over opening 328 to permit ultrasound application through the material 326. This embodiment may be advantageous in procedures where physical access to the application site is desired before and/or after application of ultrasound energy. Thus, for example, in a procedure to induce hemostasis in a blood vessel that has been accessed in a catheter procedure, after hemostasis has been achieved by application of ultrasound energy, it may be desirable to temporarily lift up the window portion of the solid acoustic couplant material 326 in order to gain access to the surface wound site (e.g., in order to insert stitches at the site).

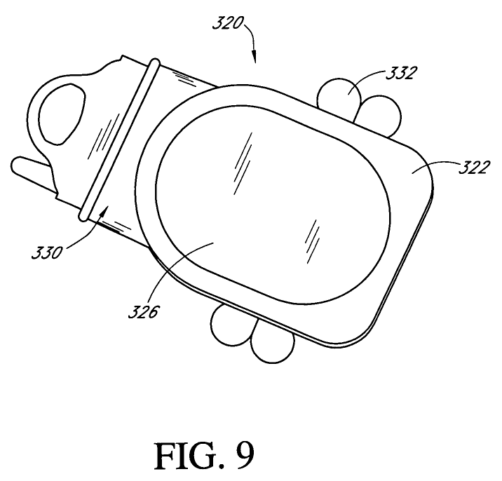

FIG. 9 illustrates another embodiment of acoustic couplant pad assembly 320. In this embodiment, housing 322 is provided along with the solid acoustic couplant material 326. The housing 322 may be coupled to a sterile receptacle means 330 to allow handling of the acoustic couplant pad assembly 320 without contacting housing 322 or solid acoustic couplant material 326. Additionally tabs 332 may be provided to enable personnel to peel away solid acoustic couplant material 326 from housing 322 without contacting the solid acoustic couplant material 326 or housing 322. Assembly 330 has a sleeve that is rolled in such a fashion that the nonsterile user can deploy the sleeve while maintaining sterility of the treatment area (for example such that the sleeve would cover and protect catheters from contamination during their being withdrawn from the patient).

In some advantageous embodiments where the acoustic couplant pad assembly is adhered to a patient, at least one surface of the solid acoustic couplant material is held in close proximity to the patient. By “close proximity,” it is meant that the solid acoustic couplant material is close enough to tissue of a patient so that ultrasonic energy may be efficiently transmitted from the couplant material into the patient, either directly or through thin layers of other material such as acoustic gel or liquid or a thin film.

As previously discussed, the acoustic couplant material incorporated within an acoustic couplant pad may either be homogenous or consist of two or more acoustic couplant materials. For example, it was previously discussed how both a solid acoustic couplant material and an acoustic gel or liquid may be incorporated within the same acoustic couplant pad assembly. Additionally, multiple solid acoustic couplant materials may be employed. For example, the solid acoustic couplant materials 250 illustrated in FIGS. 6A through 6D may consist of two or more solid acoustic couplant materials arranged in various configurations. It may be advantageous to use two or more solid acoustic couplant materials when different ultrasound transducers are employed within the same ultrasound applicator. For example, both a therapeutic ultrasound transducer array and an imaging ultrasound transducer array may be employed within the same ultrasound applicator. The optimum solid acoustic couplant material may be different for the imaging and therapeutic ultrasound transducers. The optimum solid acoustic couplant material for a particular ultrasound transducer can depend on both the acoustic properties of the ultrasound waves emitted by the transducer as well as other desired properties, such as heat transfer. In one embodiment, an acoustic couplant pad assembly is provided that comprises two or more solid acoustic couplant materials where each material is optimized to transmit ultrasonic acoustic energy of a different frequency.

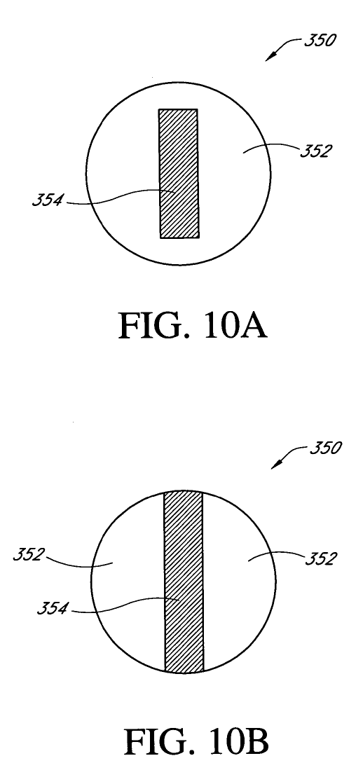

FIGS. 10A and 10B depict a plan view of a solid acoustic couplant pad 350 comprising two different solid acoustic couplant materials 352 and 354. In FIG. 10A solid acoustic couplant material 352 completely surrounds solid acoustic couplant material 354. In Figure 10B, solid acoustic couplant material 354 divides solid acoustic couplant material 352 into two sections. The embodiments of FIGS. 10A and 10B, may be advantageously used in an ultrasound applicator comprising both imaging and therapeutic ultrasound transducers. For example, solid acoustic couplant material 354 may be disposed over the imaging transducer, while solid acoustic couplant material 352 may be disposed over the therapeutic transducer array. It will be appreciated by those of skill in the art that any number of solid acoustic couplant materials and their planar configurations could be used.

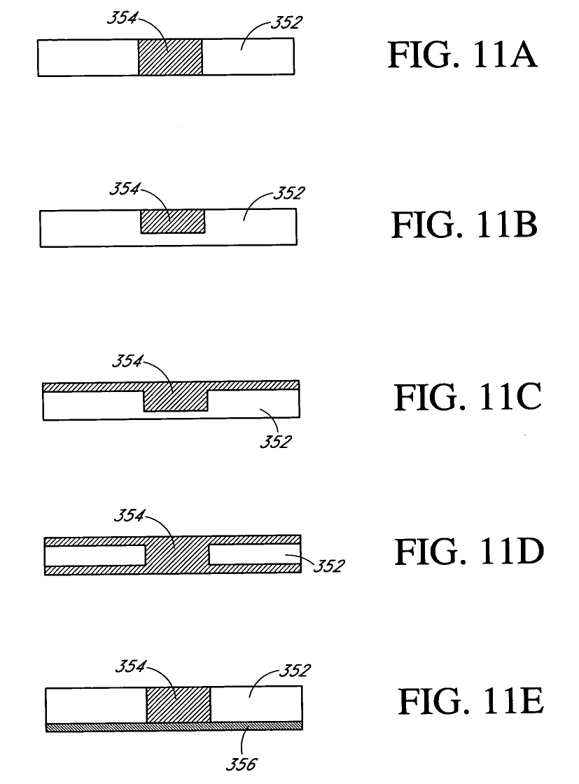

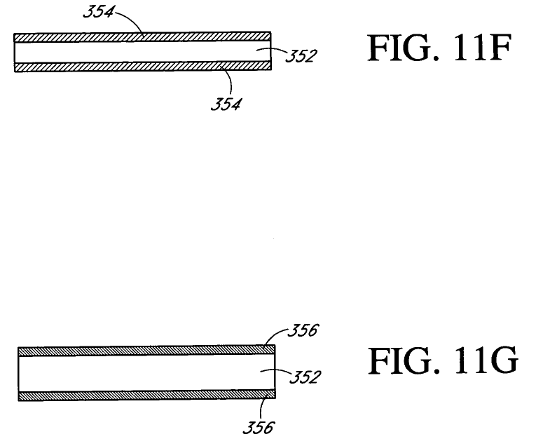

FIGS. 11A through 11G depict cross sectional views of various embodiments of acoustic couplant pads containing two or more materials. FIG. 11A depicts one configuration of solid acoustic couplant material 352 and solid acoustic couplant material 354 where solid acoustic couplant material 352 is surrounded on both sides by solid acoustic couplant material 352. It will be appreciated that the configuration in FIG. 11A may be the cross-sectional view of the configuration also depicted in FIGS. 10A or 10B. FIG. 11B depicts an embodiment where acoustic couplant material 354 does not extend all the way through acoustic couplant material 352. It will be appreciated that either the top or bottom surfaces of FIG. 11B may be used as the patient interface surface. FIG. 11C depicts an embodiment where the solid acoustic couplant material varies both in the vertical and horizontal directions. Thus, solid acoustic couplant material 354 covers one entire surface of the acoustic couplant pad as well as being disposed in part of the inner regions of the acoustic couplant pad. Again, it will be appreciated that either the top or bottom surfaces of FIG. 11C may be used as the patient interface surface. The embodiment of FIG. 11D contains acoustic couplant material 354 on both the top and bottom surfaces as well as in an inner portion of the acoustic couplant pad. However, the presence of acoustic couplant material 352 on the interior portion of the side portions of the acoustic couplant pad will vary the properties of the acoustic couplant pad on those sides. The embodiment depicted in FIG. 11E is similar to the embodiment of FIG. 11A, however one of the surfaces of the acoustic couplant pad is coated with film 356. Film 356 may or may not have acoustic coupling properties. Non-limiting examples of the film 356 are polyurethane, polyethylene, or other suitable polymers. Film 356 may be used to enhance the sterility of the acoustic couplant pad, to prevent materials in the acoustic couplant pad from interacting with patient tissue, to help provide structural integrity to the acoustic couplant pad, or to prevent the acoustic couplant pad from drying out. It will be appreciated that the film 356 may be disposed at either the patient or the ultrasound transducer interfaces. In the embodiment of FIG. 10F, acoustic couplant material 354 is located at both the patient and ultrasound transducer interfaces; however, acoustic couplant material 352 makes up the core of the acoustic couplant pad. Finally, in FIG. 10G a single solid acoustic couplant material 352 is employed, however, both surfaces of the acoustic couplant material 352 are coated with film 356, such as polyurethane, polyethylene, or other suitable polymers. The configurations of the solid acoustic couplant material disclosed herein are merely exemplary, and it will be appreciated that any number of materials and configurations may be used, depending on the combination of ultrasound transducers employed and the intended application.

As described above, acoustic gels or liquids may be employed between the solid acoustic couplant materials described herein and the patient and/or between the solid acoustic couplant materials and ultrasound transducers to eliminate or minimize the presence of air or air bubbles trapped between the interfaces that can decrease or impair acoustic transmission and efficient acoustic transfer. Any suitable commercially available ultrasound gels, liquids, and the like may be used. Preferably, the gel or liquid used is non-toxic and bio-compatible. Furthermore, various lubricating liquids, oils, and other like substances for use in conjunction with the solid acoustic couplant materials described herein may be used to facilitate or enhance movement of an acoustic transducer across the transducer contacting surface of the solid acoustic couplant material or movement of the solid acoustic couplant material across the surface of a patient. The lubricating substance may be any conventionally available ultrasound or scanning gels, liquids, or the like. In one possible implementation, ScanLube® available from Sonotech, Inc. of Bellingham, Wash. can be employed. Advantageously, the lubricating means is non-toxic, an efficient acoustic transmitter, and non-degrading, and has acoustic properties similar to the solid acoustic couplant material in contact with it. In addition to promoting movement across surfaces, the lubricating substance may facilitate intimate contact between the solid acoustic couplant media and the ultrasound transducer and/or the patient, thereby minimizing or eliminating effects of any air bubbles that may be present that can disperse the emitted acoustic waves and result in sound wave deterioration.

As previously discussed, acoustic gel or liquid may be either pre-disposed on the transducer and/or patient surfaces of a solid acoustic couplant material or manually applied prior to use. Alternatively, a reservoir of acoustic gel or liquid may be incorporated within an acoustic couplant pad assembly, such as those described above, so that the gel or liquid may be dispensed from the reservoir onto the patient and/or transducer surfaces of the acoustic couplant material just prior to use. In some embodiments, the reservoir may be incorporated within the housing holding the acoustic couplant material. Additionally, ports may be provided in the housing so that the gel or liquid is dispensed through the ports in the housing directly onto the patient and/or transducer surfaces of the solid acoustic couplant material.

Solid Acoustic Couplant Materials

The solid acoustic couplant materials for use as described herein advantageously have the properties of permitting efficient transmission of ultrasound energy through them. More specifically, it is advantageous that the acoustic impedance and velocity be matched to the tissue to which the ultrasound energy is to be transmitted. It is also advantageous for the solid acoustic couplant materials to provide a sterile barrier. In some embodiments, particularly where HIFU ultrasound energy is to be used for therapeutic use, it may be desirable that the solid acoustic couplant materials provide a thermal insulative barrier between the ultrasound transducers and the skin of the patient. Other properties that may be desirable in the solid acoustic couplant materials are that they be soft, flexible, and conformal so that the interfaces between the ultrasound transducer and the surface of the patient do not have any intervening air bubbles, which-could interfere with the transmission of ultrasound energy. Furthermore, it may be desirable for the solid acoustic couplant material to have high tensile strength and elongation properties as well as be lubricious, transparent, nontoxic, odorless, and easy to manufacture. Finally, when the solid acoustic couplant material is to be used in a hemostasis procedure, it may be desirable that the material have a robust compression force transmission so that a user may apply compression force to the wound site in order to temporarily stop bleeding prior to application of the ultrasonic energy.

In some embodiments, the solid acoustic couplant material advantageously is gelatinous. As used herein, “gelatinous” refers to material having the property that it may be compressed and/or stretched while substantially returning to its original shape after the compression or stretching forces are removed. In some embodiments, the solid acoustic couplant material for use as described herein is a hydrogel. Such materials are described, for example, in U.S. Pat. No. 6,039,694, which is incorporated herein by reference in its entirety. In some embodiments the solid acoustic couplant material is a thermoplastic elastomer (TPE). Some TPEs and methods for making them are described in U.S. Pat. No. 5,994,450, which is incorporated herein by reference in its entirety. When a plurality of solid acoustic couplant materials are employed, such as described above, the various materials may include both variation in type, such as hydrogels and TPEs, as well as variation in composition within a single type, such as multiple compositions of TPEs.

In some embodiments, TPEs are particularly useful for use with HIFU therapeutic ultrasound. When compared to hydrogels, TPEs have the improved properties of being better thermal insulators and not drying out, and thereby maintaining lubricity. These properties owe to the fact that TPEs employ oil to enhance their softness instead of the high water content incorporated within hydrogels. Furthermore, TPEs can be easily manufactured in a variety of shapes using molds or extrusion. In contrast, hydrogels may be better suited for use with imaging transducers because the high water content of hydrogels make them good acoustic couplers and a heat stand-off may not be required with an imaging transducer.

In one embodiment, the present invention provides methods, devices, compositions, and systems for the novel use of gelatinous thermoplastic elastomers (“TPEs”) as an acoustic transmission media during diagnostic and therapeutic (HIFU) ultrasound applications. As used herein, “gelatinous TPEs” refers to oil-enhanced TPEs where oil is used to enhance softness or TPEs containing a resin to enhance softness.

In one embodiment, TPEs for use as described herein comprise a di-block or a tri-block copolymer configuration comprising a hard block segment, a soft block segment, and which may or may not contain a softness enhancing oil. The advantages and inherent properties of thermoplastic elastomeric compositions that make these materials suitable for use as an acoustic transmission media are many. For example, TPE's can be oil-extended to produce soft, flexible, conformal, and gelatinous compositions exhibiting the following properties: high dimensional stability; crack, tear, and creep resistance; excellent tensile strength; high elongation properties; low thermal conductivity; long service life under stress; excellent processing ability for cast molding; non-toxicity; nearly odorless; extremely soft yet strong and capable of being repeatedly handled, and possessing elastic memory with substantially little or no oil bleedout. TPEs can also be configured to be transparent and can be configured to be sterilized by conventional methods, including but not limited to: gamma radiation, e-beam, gas and (steam, dry) heat sterilization.

In one embodiment of the present invention, the di-block copolymers of the present invention have the general configuration A-B, wherein A is the hard block segment and B is a soft block segment. In another embodiment, the tri-block copolymer of the present invention has the general configuration A-B-A. In one embodiment, the hard block segment A comprises a polymer of a monoalkylarene. In one embodiment, the soft block segment B comprises a polymer of an aliphatic hydrocarbon. In one embodiment, the soft block segment B comprises a diene. In one embodiment, A is polystyrene and B is an elastomeric high molecular weight segment that may be comprised of, for example, polymers of the following: butadiene (B), isoprene (I), isoprene-butadiene (IB), ethylene-butylene (EB), ethylene-propylene (EP), ethylene-butylene-ethylene-propylene (EBEP), or ethylene-ethylene-propylene (EEP). In some embodiments, the soft block polymer materials are hydrogenated. In some embodiments, mixtures of soft block components may be used. These soft segments are provided as an example only and are not intended to be limiting. Other soft segments known in the art can be incorporated into the di-block or tri-block polymers of the present invention. Similarly, other hard block segments known in the art can be used. For example, poly(methyl-methacrylate) may be used for the hard block segment instead of polystyrene.

Various other components may be added to the polymers disclosed herein. For example, additives that modify the physical properties of the TPE may be included. In one embodiment, soft block compatible modifiers (e.g., modifiers that mix well with the soft block component) may be added. In another embodiment, hard block compatible modifiers (e.g., modifiers that mix well with the hard block component) may be added. Examples of soft block compatible modifiers are softness enhancing oils or resins. Another example is polypropylene, which may be added to increase strength, rigidity and to reduce oil bleed from oil enhanced TPEs. Hard block compatible modifiers may be added to modify thermal properties of the TPE, such as increasing the melting point or glass transition temperature (T_{g}) in order to increase the thermal stability of the TPE and provide greater heat insulative properties. In addition, hard block compatible resins may be included to enhance softness. Non-limiting examples of hard block compatible modifiers include low molecular weight polystyrene homopolymer, polyphenylene oxide, and resins such as Noryl® PPO available from GE Plastics. Any of the many soft block and hard block modifiers known in the art may be included in the TPEs disclosed herein. Other modifiers that may be included in TPEs include detackifiers, antioxidants, flame retardants, colorants, and odorants, such as described in U.S. Pat. No. 5,994,450, which is incorporated herein by reference in its entirety.

In one embodiment, the average polymer block molecular weights are between 5,000 to 75,000 for the hard polymer blocks and between 25,000 and 250,000 for the soft polymer blocks. In one embodiment, the average block molecular weights are between 8,000 to 65,000 for the hard polymer blocks and between 35,000 and 110,000 for the soft polymer blocks. The polymer materials may be commercially available, such as those available from Shell under the Kraton® or Septon® designation from Kuraray Co. Ltd. It will be understood that the block polymers may comprise more complicated structures of either linear or branched configurations and may contain any desired number of polymer blocks.

In one embodiment, pre-synthesized gelatinous TPEs for use as described herein may be obtained from commercials sources, such as Gelastic™ available from Edizone, LC (Alpine, Utah) or gels available from Silipos®, Inc. (New York, N.Y.). In some embodiments, pre-synthesized gelatinous TPEs may be modified by adding modifiers such as additional amounts of oil or any of the soft-block or hard-block modifiers disclosed herein. Such additives may be introduced by melting the commercially obtained TPEs, adding the additional material, and cooling the modified TPE in the desired shape. In one example, a SEEPS TPE containing 86% mineral oil was obtained from Silipos®, Inc. The TPE was melted and varying amounts of Drakeol® 34 mineral oil (Penreco, Karns City, Pa.) was added to increase the oil content of the TPE.

In some embodiments, the TPEs as described herein can be used for both diagnostic and/or therapeutic ultrasound applications. Typically, TPE or gelatinous TPE and articles made therefrom, will be disposed between a patient 110 and an ultrasound transducer 104 as illustrated in FIG. 1. Various TPE articles can be used on a patient's skin, against organs and/or tissues, or inside a body cavity.

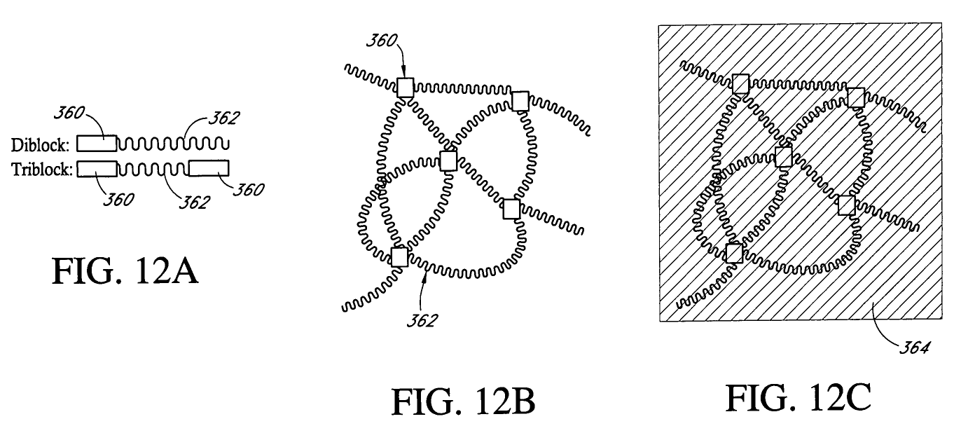

FIGS. 12A through 12C depicts the chemical composition of a typical TPE. As depicted in FIG. 12A, TPEs may advantageously have a di-block or triblock structure. Both structures contain a hard block component 360 and a soft block component 362. The hard block component 360 act as a cross-linking point at a temperature below the glass transition temperature (T_{g}) of the hard block component 360. In one embodiment, the hard block component 360 comprises polystyrene. The soft block component 362 acts to provide rubber-like properties. Hydrogenation of the soft block component provides excellent heat resistance and weatherability. As depicted in FIG. 12C, the TPE may be softened by incorporating an oil and/or resin 364 within the polymer matrix. Additionally, other soft block modifiers and hard block modifiers may be included.

One method of making TPE or gelatinous TPE compositions suitable for use according to the present invention is as follows. First, di-block or tri-block copolymers as described above and any additives are heat blended to from an admixture. By heat blending, it is meant that the mixture is heated to melting while agitating the mixture. Advantageous heat blending temperatures are between 260° F. and 290° F. Advantageous melting times include 10 minutes or less, five minutes or less, and 90 seconds or less. The second step of TPE synthesis involves adding a heated oil to the copolymers and heat blending the composition. In one embodiment, 2 to 15 parts by weight of oil to 1 part by weight of copolymer is added. In one advantageous embodiment, the oil composition and amount is such so as to provide compositions that can be softened or melted at elevated temperatures but which regain elastomeric properties at ambient temperatures. In one embodiment, all components of the TPE are mixed in one step and then quickly heated to melting. The final step of TPE synthesis comprises forming a cast of the TPE by pouring the heated admixture composition into a mold to shape the TPE material. Upon cooling and removal from the mold, the TPE cast will retain its shape. Alternatively, the TPE material may be extruded or other suitable shaping techniques may be utilized.

In another embodiment, TPEs for use as described herein are synthesized by dissolving the block copolymer components in a solvent, adding the oil or resin and any other additives, and then removing the solvent from the mixture.

Suitable TPE compositions can be prepared by using di-block or tri-block copolymer components, as provided in Table 1 and as further described in U.S. Pat. Nos. 5,994,450; 6,117,119; and 6,673,054, the entire contents of which are hereby incorporated herein by reference. As described in these patents, admixtures of the copolymers are advantageously heated to about 150° C. In the TPE designations of Table 1, S refers to a polystyrene hard block segment.

TABLE 1
TPE Designations and Compositions

| | TPE | Hard Block Segment (Styrene) | |
|---|---|---|---|
| | Designation | Exemplary Styrene | Preferred Styrene |
| Soft Block | (Triblock, | Content | Content |
| Segment | Diblock) | (Weight percent) | (Weight percent) |
| Butadiene (B) | SBS, SB | 5-60 wt. % | 15-25 wt. % |
| Isoprene (I) | SIS, SI | 5-60 wt. % | 15-25 wt. % |
| Ethylene-butylene | SEBS, SEB | 5-60 wt. % | 15-25 wt. % |
| (EB) | | | |
| Ethylene- | SEPS, SEP | 5-60 wt. % | 15-25 wt. % |
| propylene (EP) | | | |
| Ethylene- | SEEPS, | 5-60 wt. % | 15-25 wt. % |
| ethylene- | SEEP | | |
| propylene (EEP) | | | |

To form a gelatinous TPE, various oils can be added as a softening agent to the various di-block or tri-block compositions provided above. Exemplary oils that can be employed for this purpose are provided in Table 2. For the oils identified in Table 2, the Chemical Abstract System (CAS) numbers or Registry Numbers and synonyms are provided.

Various mineral oils, including the following can also be employed as the softening oil of the present invention: paraffin oils; napthalenic oils; adepsine oil; alboline; bayol 55; bayol f; blandlube; blandol® white mineral oil; cable oil; carnea® 21; clearteck; crystol 325; crystosol; drakeol®; electrical insulating oil; ervol®; filtrawhite; fonoline®; fligol; Gloria®; glymol; heat-treating oil; hevyteck; hydraulic oil; hydrocarbon oils; jute batching oil; kaydol®; kondremul®; kremol®; lignite oil; liquid paraffin; lubricating oil; mineral oil, paraffinic; mineral oil, aromatic; mineral oil hydrocarbon solvent (petroleum); mineral oil mist; mineral oil (saturated paraffin oil); mineral seal oil; Molol; neo-cultol®; Nujol; oil mist; OIL MIST, MINERAL (MINERAL OIL); oil mist, mineral, severely refined; oil mist, refined mineral; oil, petroleum; paroleine; peneteck®; penreco®; perfecta®; petrogalar; petrolatum, liquid; Petroleum hydrocarbons; primol®; primol® 355; primol® d; protopet®; Saxol; tech pet f; triona b; Uvasol; white mineral oil; and white oil. Other oils having similar chemical and physical properties as those identified herein can also be used and are within the scope of the present invention. Preferably, the oil content of the resulting gelatinous TPE can range from about 0-95% wt. Moreover, the preferred softening oils should be compatible with the soft-block segments but not the hard-block styrene segments.

TABLE 2
Plasticizing Oils

| CAS Number | Plasticizing Oil |
|---|---|
| [64742-52-5] | Hydrotreated heavy naphthenic distillate. Synonyms: |
| | Distillates (petroleum), hydrotreated heavy naphthenic; |
| | hydrotreated heavy naphthenic distillate. |
| [64742-18-3] | Acid-treated heavy naphthenic distillate. Synonyms: |
| | acid-treated heavy naphthenic distillate. |
| [8042-47-5] | Light and heavy mineral oil. Synonyms: Mineral oil, |
| | light and heavy; White mineral oil, petroleum. |
| [64741-96-4] | Solvent-refined heavy naphthenic distillate. Synonyms: |
| | Distillates (petroleum), solvent-refined heavy naphthenic; |
| | solvent-refined heavy naphthenic distillate. |
| [64742-54-7] | Hydrotreated heavy paraffinic distillate. Synonyms: |
| | Distillates (petroleum), hydrotreated heavy paraffinic; |
| | distillates (petroleum), hydrotreated heavy paraffinic; |
| | hydrotreated heavy paraffinic distillate. |

Moreover, the composition of the TPE or gelatinous TPE disclosed herein can also contain other soft block compatible modifiers and hard block compatible modifiers as well as small amounts of conventionally employed additives such as stabilizers, antioxidants, anti-blocking agents, colorants, fragrances, and the like to an extent not affecting or decreasing the desired properties of the present invention.

In one specific example of a gelatinous TPE, one part SEPTON 4055 from Kuraray (an ultra high molecular weight polystyrene-hydrogenated poly(isoprene+butadiene)-polystyrene triblock copolymer) and eight parts LP 150 mineral oil were compounded in an ISF 120VL injection molding machine. The temperature was increased stepwise from the point of insertion to the injection nozzle. At the point of insertion, the temperature was about 270° F. Temperatures along the screw were about 275° F. and about 280° F., with the temperature increasing as the material approached the injection nozzle. The temperature at the injection nozzle was about 290° F. The composition was then injected into an aluminum plaque mold and allowed to cure at room temperature for about 24 hours.

It will be appreciated by one skilled in the art that various TPE or gelatinous TPE compositions can be optimized in order to vary the mechanical and/or acoustic properties of these materials. For example, a TPE formulation with less oil will have a higher durometer or compressive strength (or will be harder) and will have a lower elongation (less stretchable). An optimization strategy or technique may include: evaluate various oils that are commonly used and select the oil with the best impedance and attenuation properties for a particular transducer to be used. Yet another technique may be to evaluate and characterize the various soft-block segments provided herein at a control hard block component content to find the oil and soft-block combination producing the best mechanical and/or acoustic properties for a particular ultrasound procedure or application. In another embodiment, various soft block and hard block modifiers are added to a control TPE to adjust the thermal, physical, and/or acoustic properties of the TPE. In yet another possible implementation, different hard block to soft block ratios and molecular weights of these components can also be varied to optimize TPE compositions to achieve the desired mechanical and/or acoustic characteristics.

Thus, in one embodiment, a method of optimizing a TPE composition for use as an acoustic coupler is provided. In this embodiment, the soft block segment, oil, hard block modifier, and/or their respective composition in the TPE may be varied to alter or optimize the acoustic (e.g. acoustic impedance or attenuation properties) and/or physical properties of the TPE to a desired level.

Gel or Liquid Acoustic Coupling Interfaces

In some embodiments, an acoustic couplant pad is provided that comprises a gel or liquid acoustic couplant material incorporated within a form retaining housing. Such an acoustic couplant pad may be used in any of the embodiments of the solid acoustic couplant material pads described. The gel or liquid acoustic couplant material may be any gel or liquid having sufficient acoustic coupling properties, such as the many commercial gels or liquids currently available for ultrasound applications. The form retaining housing may be any suitable material for retaining the gel or liquid. In one advantageous embodiment, the form retaining housing may be flexible and is thin enough so as not to interfere with efficient transmission of acoustic energy through the acoustic couplant pad. In one embodiment, the form retaining housing may itself be an efficient acoustic couplant. Non-limiting examples of a material for the form retaining housing are polyurethane, polyethylene, or other suitable polymers.

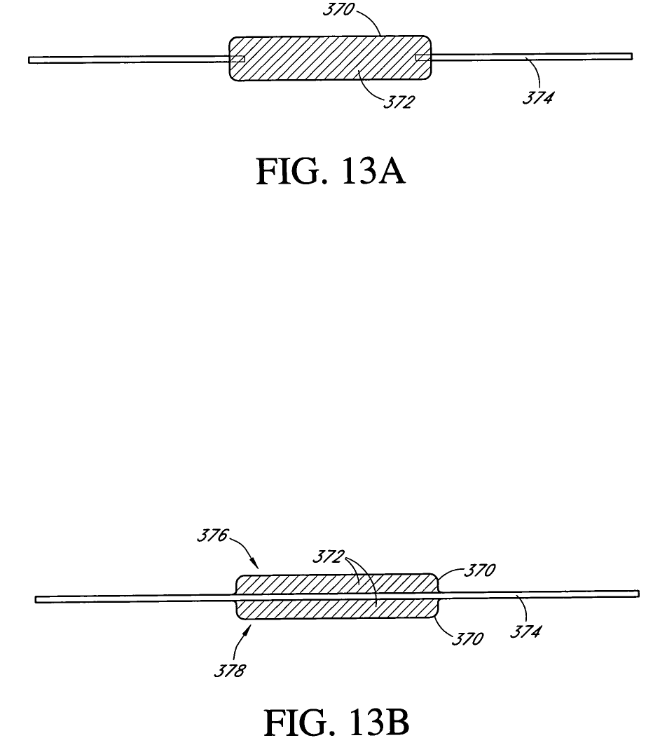

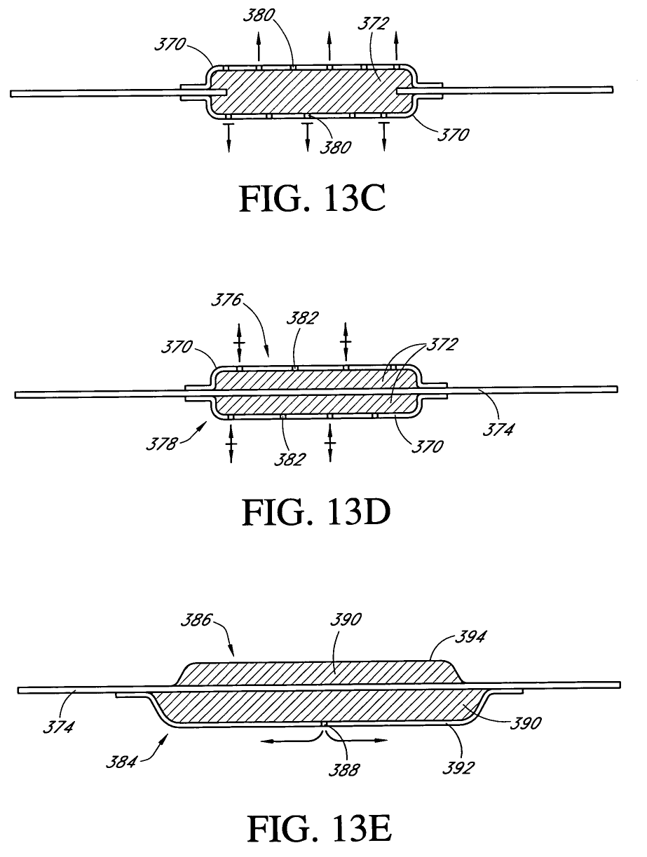

Cross-sectional views of several non-limiting embodiments of acoustic couplant pads containing a gel or liquid acoustic couplant material is depicted in FIGS. 13A through 13E. In FIG. 13A, form retaining housing 370 contains gel or liquid 372. Form retaining housing 370 forces gel or liquid 372 to substantially maintain a desired shape, however, it may generally still be pliable so as to allow the acoustic couplant pad to conform to the shape of ultrasound transducers and/or a patient. In one advantageous embodiment, form retaining housing 370 also provides a sterile barrier to prevent passage of microbes from one side of the acoustic couplant pad to the other. In one embodiment, the form retaining housing 370 and gel or liquid 372 may be coupled to an ultrasound applicator such as described above for solid acoustic couplant pads. In another embodiment, the form retaining housing 370 and gel or liquid 372 may be coupled to a patient or freely disposed between an ultrasound applicator and a patient. In one embodiment, the housing 370 may be coupled to a sterile barrier 374 such that the combined housing 370 and sterile barrier 374 provide a continuous sterile barrier between a patient and an ultrasound applicator while still providing the acoustic coupling properties of gel or liquid 372. Sterile barrier 374 may be any material suitable for preventing passage of microbes from one side to the other. The sterile barrier 374 may advantageously be flexible and substantially transparent. In one embodiment, the sterile barrier 374 is a polyurethane, polyethylene, or other suitable polymer film.

In one embodiment, depicted in FIG. 13B, the sterile barrier 374 may be continuous such that one acoustic couplant pad, 376 and 378, are disposed on each side of the sterile barrier 374. Each acoustic couplant pad comprises a form retaining housing 370 in which is disposed a gel or liquid 372. Advantageously, the sterile barrier 374 is then thin enough so as not to impede the transmission of acoustic energy from acoustic couplant pad 376 to acoustic couplant pad 378 or alternatively is itself constructed of an acoustic coupling material.

In one embodiment, depicted in FIGS. 13C and 13D, the form retaining housing 370 may comprise ports 380 and 382 that allow for the deployment of gel or liquid 372 to the outside of the acoustic couplant pad. Such deployment allows for gel or liquid 372 to be disposed between the form retaining housing 370 and the surface of a patient on one side, and between the form retaining housing 370 and ultrasound transducers on the other. Thus, any air between the form retaining housing 370 and the patient and/or ultrasound transducers may be excluded by displacement with gel or liquid 372. Furthermore, if any air bubbles form within gel or liquid 372, the bubbles may be pushed out of ports 380 or 382. In some embodiments, deployment of gel or liquid 372 through ports 380 or 382 may be achieved by applying pressure to the acoustic couplant pads. In some embodiments, a peel away cover may be disposed over the ports 380 and 382 prior to use, thereby preventing leakage of gel or liquid 372 until the acoustic couplant pad is ready for use. In one embodiment, depicted in FIG. 13C, a single acoustic couplant pad is used with one-way ports 380. The use of one-way ports 380 allow gel or liquid 372 to deploy out of the acoustic couplant pad but prevents microbes from entering through ports 380 and exiting on the opposite side. Thus, the acoustic couplant pad maintains a sterile barrier between a patient and an ultrasound applicator. In another embodiment, depicted FIG. 13D, two-way ports 382 may be employed, however, the sterile barrier 374 is continuous such that there are two separate acoustic couplant pads, 376 and 378. Thus, although microbes may enter acoustic couplant pads 376 and 378, sterile barrier 374 prevents passage of microbes from one acoustic couplant pad to the other. Thus, a sterile barrier is maintained between an ultrasound applicator and a patient.

In another embodiment, depicted in FIG. 13E, two acoustic couplant pads, 384 and 386 are provided on opposite sides of a sterile barrier 374. Acoustic couplant pad 384 comprises one or more ports 388 that allow gel or liquid 390 to be deployed so that it is disposed between form retaining housing 392 and a patient or an ultrasound applicator. Acoustic couplant pad 386 does not contain any gel deploying ports. Thus, the interface between form retaining housing 394 and a patient or an ultrasound applicator may either have sufficient acoustic coupling without intervening gel or liquid or the gel or liquid at the interface may be manually applied prior to use.

It will be appreciated by those of skill in the art that other configurations of gel or liquid disposed within a form retaining housing than those discussed above may be employed. For example, multiple housings with different shapes and configurations may be used, for example, employing multiple housings on the same side of sterile barrier 374. In addition, multiple configurations and types of gel or liquid deploying ports may be employed. In some embodiments, gel or liquid may be deployed through a form retaining housing that is uniformly semi permeable to the gel or liquid. Finally, gel or liquid based acoustic couplant pads may be combined with the solid acoustic couplant pads described above. Thus, for example, the patient side of an acoustic couplant pad assembly may employ a gel or liquid based acoustic couplant pad while the ultrasound transducer side may employ a solid acoustic couplant pad. Any number of operable combinations of acoustic couplant pads and materials are possible.

Pre-sterilized Patient Interface

In some embodiments, a presterilized sterile barrier is provided for deployment around medical instrumentation prior to use of the instrumentation on a patient. Use of such a presterilized sterile barrier eliminates the need for medical personnel to sterilize the medical instrumentation or the sterile barrier prior to commencing the procedure. Thus, such a presterilized barrier reduces the preparation time for certain medical procedures. In one embodiment, the presterilized sterile barrier is configured so that it may be deployed around the medical instrumentation by nonsterile personnel. One such embodiment is depicted in FIG. 13. In this embodiment, a flexible sheath 400 is provided that is adapted to prevent passage of microbials through the sheath. The flexible sheath 400 resembles a bag. An openable seal 402 is provided at the open end of the bag. When the seal 402 is closed, it provides a barrier to microbes from entering the inner portion 406 of the sheath 400. The seal 402 may be designed to be opened and resealed or to be only opened once. The seal 402 may consist of an adhesive such as the tape, a seal structure such as a Ziploc, or may be incorporated into the sheath 400 itself, such as by heat sealing the sheath material. Additionally, a piece of material (e.g., Tyvek®) can be attached so as to form an openable seal. The material of the sheath 400 may be any suitable flexible microbial resistant material such as polyurethane, polyethylene, or other suitable polymer. In some embodiments it is advantageous that the sheath material 400 be transparent so that medical personnel can visualize the medical instrumentation through the sheath 400.

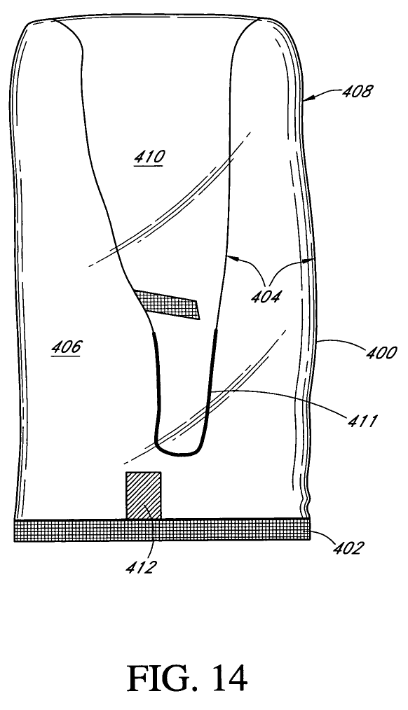

The inside surface 404 of the sheath 400 may be presterilized prior to closure of seal 402. Sterilization of the sheath 400 may be by any suitable technique such as dry heating, steam sterilization, ethylene oxide (ETO) treatment, or electron beam or gamma radiation. Upon sterilization, seal 402 is closed and then the presterilized sterile barrier may be distributed to medical personnel in a predeployed state. Thus, in the predeployed state the interior volume 406 and interior surface 404 of the sheath 400 is sterile while outside surface 408 is nonsterile. The sheath 400 may be partially placed inside out as depicted in FIG. 14 so as to create an instrument cavity 410. The sheath 400 may be placed in this configuration either by medical personnel or be prepackaged in this configuration. Medical personnel can then place the desired instrument within cavity 410. In some embodiments, a portion 411 of the sheath 400 may be made more rigid than the rest of the sheath 400 in order for it to conform more tightly around the shape of the medical instrument. Finally, seal 402 can be opened and the sheath 400 completely placed inside out to obtain the post deployed state. Opening of the seal 402 enables the medical instrument to be passed through the open end of sheath 400 (i.e., by completely inverting sheath 400 inside out). In the post deployed state, surface 404 is now on the outside, while surface 408 faces towards the medical instrument. Thus a sterile outside surface 404 is presented for contact with the patient. One or more tabs 412 may be provided to enable medical personnel to open seal 402 and invert the sheath 400 over the medical instrument without compromising the sterile field within sheath 400. Thus, the sheath 400 may be applied over a medical instrument by nonsterile personnel.

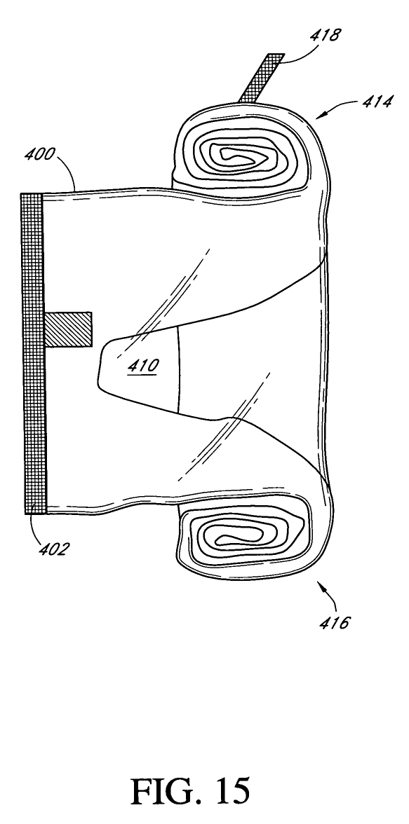

In one embodiment, depicted in FIG. 15 sheath 400 is prepared as in FIG. 14 with the sheath partially placed inside out. However, the doubled over sheath 400 may be rolled to create roles 414 and 416 in order to make the predeployed sheath 400 more compact. Rolls 414 and 416 may be secured using tape or some other adhesive. Tab 418 may be provided to help medical personnel handle the predeployed sheath 400 when inserting a medical instrument within cavity 410. Prior to breaking of seal 402 and complete inversion of sheath 400 over the medical instrument, the roles 414 and 416 may be unfurled so that the sheath 400 has the shape as depicted in FIG. 14.

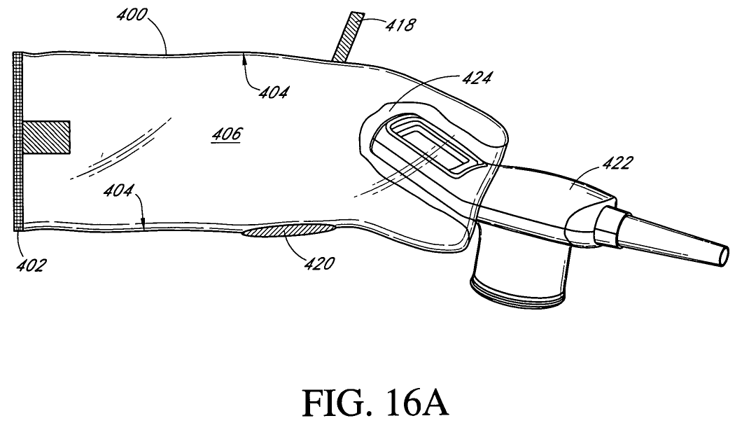

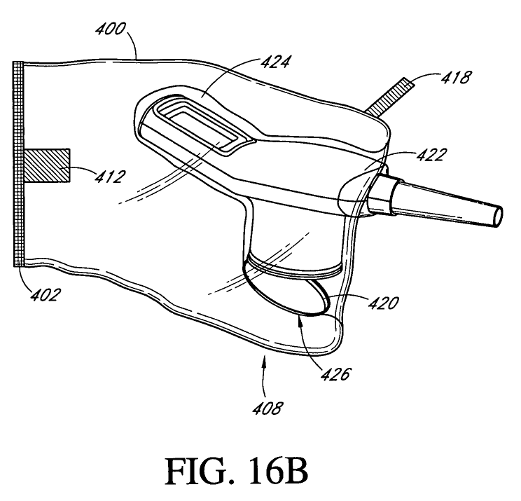

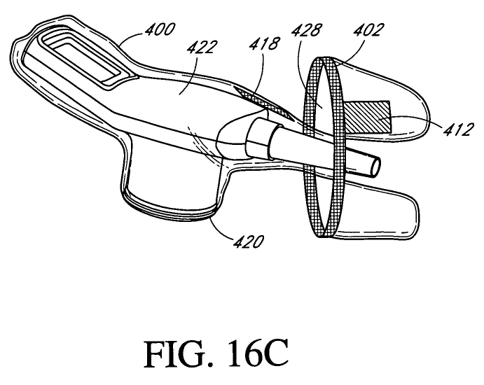

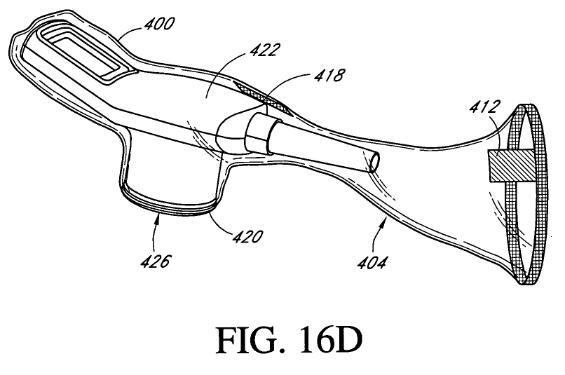

In some embodiments devices may be incorporated within the surface of sheath 400 so as to interface with features on the medical instrument. For example various access portals may be deployed on the surface of the sheath 400. One embodiment, depicted in FIGS. 16A through 16D illustrate the incorporation of an acoustic couplant pad assembly within the surface of sheath 400. As depicted in the figures, sheath 400 is provided with an acoustic couplant pad assembly 420 incorporated within the surface of the sheath. The acoustic couplant pad assembly 420 may include any acoustic couplant pad or pads, such as any of the structures described above. As illustrated in FIG. 16A ultrasound applicator 422 may be partially inserted within a cavity 424 within sheath 400. The sheath 400 contains presterilized inner cavity 406 and inner surface 404. Using tab 418, the sheath 400 may be pulled toward the ultrasound applicator 422 so that larger portions of ultrasound applicator 422 is disposed within cavity 424. As depicted in FIG. 16B, acoustic couplant pad assembly 420 may be snapped onto ultrasound applicator 422 prior to opening of seal 402. The acoustic couplant pad assembly 420 may be coupled to the ultrasound applicator 422 by contacting nonsterile surfaces 408, thus protecting the sterility of the patient interfacing surface 426 of the acoustic couplant pad assembly 420. It will be appreciated that this coupling procedure may be performed by nonsterile personnel. Next, as depicted in FIG. 16C, seal 402 may be opened to create opening 428. This opening may then be passed over the ultrasound applicator 422 to further deploy sheath 400 around the applicator 422. Seal 402 may be opened and the opening 428 passed over the applicator 422 using tab 412 to prevent nonsterile contact with the opening 428. Finally, as depicted in FIG. 16D. The sheath 400 may be completely inverted so that ultrasound applicator 422 is completely contained within the sheath 400. Thus, the sterile surface 404 is now exposed to the outside and provides a sterile barrier between the patient and the ultrasound applicator 422, as well as between the patient and other relevant parts of an ultrasound applicator such as its RF power and control cable. As described earlier, the acoustic couplant pad assembly 420 may optionally comprise a removable thin barrier on the patient interface surface 426 that can be removed as a last step prior to commencing with the ultrasound procedure.

It will be appreciated that structures other than acoustic couplant pad assembly 420 may be implemented with a sterile barrier 400 such as depicted in FIGS. 16A through 16D for use with a variety of medical instrumentation. In some embodiments, features such as acoustic couplant pad assembly 420 are not incorporated within sterile sheath 400 but are rather secured after sterile sheath 400 is deployed around the medical instrument. Thus for example, acoustic couplant pad assembly 420 may be secured as depicted in FIG. 16D with the sheath 400 disposed between the acoustic couplant pad assembly 420 and the ultrasound applicator 422. In such an embodiment, it may be advantageous to apply an acoustic gel or liquid between both the sheath 400 and the ultrasound applicator 422 and between the acoustic couplant pad assembly 426 and the sheath 400 to promote acoustic coupling.

Sterile Barrier for Isolation of a Site and/or Instrument

In one embodiment, a sterile barrier is provided around a body site, such as a wound site, and/or medical instrumentation that advantageously are to be kept in a sterile environment. Such a sterile barrier isolates the desired site on a patient, allowing non-sterile personnel and instrumentation to be used at other body sites on the patient without risk of contaminating the sterile site. In one embodiment, the sterile barrier is adhered to the surface of a patient, thus providing a sterile seal between the patient and the sterile barrier.

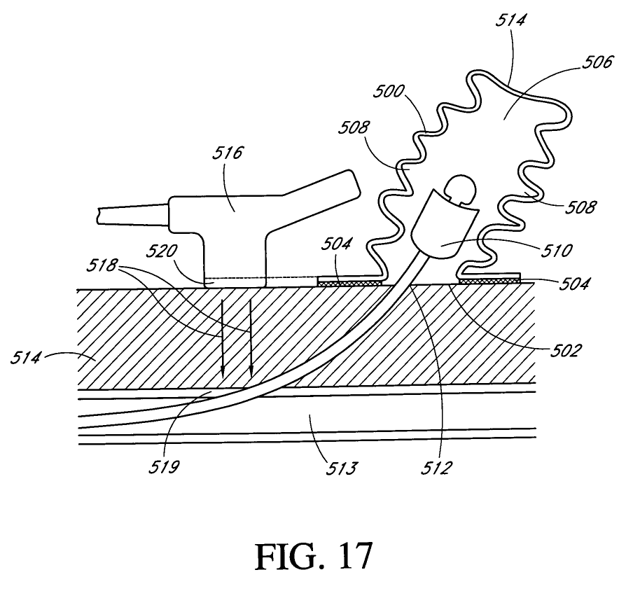

One embodiment, depicted in FIG. 17, provides a sterile barrier 500 around a catheter insertion site 502. The sterile barrier 500 is adhered to the patient's skin using adhesive 504, thus providing a sterile seal between the sterile barrier 500 and the patient's skin. The sterile barrier 500 then provides a sterile cavity 506 that is protected against contamination from outside the sterile barrier 500. In one embodiment, sterile barrier 500 is provided with a protective film disposed on adhesive 504 prior to use. The protective film may serve the function of protecting the adhesive 504 prior to use and maintaining the sterility of cavity 506 prior to use. Thus, the sterile barrier 500 may be provided to a user in a pre-sterilized state with sterile cavity 506 protected by the protective film. Prior to use, the user may peel off the protective film and then adhere the sterile barrier 500 to the desired site.

In one embodiment, the sterile barrier 500 comprises flexible material to allow manipulation of medical instruments disposed within cavity 506. In one embodiment, sterile barrier 500 comprises folds or bellows 508 that allow sterile barrier 500 to elongate. Thus, for example, the introducer sheath 510 for a catheterization procedure may be removed from blood vessel 513 and patient tissue 514 by grabbing the sheath 510 through the sterile barrier 500 and pulling the sheath 510 out of the patient. While pulling the sheath 510, the sterile barrier 500 can elongate, thereby facilitating removal of the sheath 510 without compromising the sterility of the resulting wound site 512. Pressure may be applied to the wound site 512 through sterile barrier 500 to temporarily stop blood flow through the wound site 512. Those of skill in the art will appreciate multiple structures and materials for allowing elongation and manipulation of sterile barrier 500. Furthermore, those of skill in the art will appreciate that instruments other than an introducer sheath 510 may be utilized within sterile cavity 506. For example, sterile barrier 500 may be used to perform surgical procedures with a surgical instrument disposed within cavity 506. Furthermore, introducer sheaths other than that depicted in FIG. 17 may be used, such as sheaths with side ports or other attachments.

In some embodiments, sterile barrier 500 is provided with features that allow access to cavity 506. For example, various ports may be disposed within sterile barrier 500. In one embodiment, a removable cap is provided on end 514 allowing access to cavity 506. The cap may be any suitable structure. In one embodiment, the cap is a peel-off structure disposed over an opening in end 514.

One advantage of sterile barrier 500 is that after it is adhered to the patient, non-sterile personnel may manipulate instruments disposed within cavity 506 without contaminating would site 512. Furthermore, non-sterile instruments may be used outside of sterile barrier 500 without risk of contamination. Thus, for example, a non-sterile therapeutic ultrasound applicator 516 may be placed on the patient's skin in order to supply ultrasound energy 518 to effect sealing of the walls 519 of blood vessel 513 where introducer sheath 510 has pierced the walls. Because the wound site 512 is separated from the ultrasound applicator 516 by sterile barrier 500, the ultrasound applicator 516 need not be sterile. Thus, for example, traditional ultrasound gels or liquids may be used between the ultrasound transducers in the ultrasound applicator 516 and the patient without need of a sterile barrier between the applicator 516 and the patient. In one embodiment, an access port or cap as discussed above may be used to introduce a targeting aid or to flush wound site 512 during the ultrasound procedure.

In one embodiment, an acoustic couplant pad 520, such as any of the pads discussed above, may be disposed between the ultrasound applicator 516 and the patient. In one embodiment, the acoustic couplant pad 520 is coupled to the sterile barrier 500. Thus, when the sterile barrier 500 is adhered to the patient, an acoustic couplant pad 520 is provided to facilitate use of ultrasound applicator 516. The combination apparatus of acoustic couplant pad 520 and sterile barrier 500 may be provided as a convenient disposable single article.

ID and History Tracking of Patient Interfaces

In some embodiments, devices and methods for identifying the patient interface and/or tracking the history of a patient interface is provided. Such identification and tracking may be accomplished by incorporating an ID tag on the patient interface. In some embodiments the ID tag only provides a unique identifier of the patient interface. In other embodiments the ID tag also provides a means for recording and tracking the history of the patient interface. In one embodiment, the ID tag comprises a bar-code that may be optically scanned by an optical scanner incorporated within the medical instrument with which the patient interface is to be used. Thus, when the patient interface is brought within proximity of the medical instrument, the medical instrument can scan the bar-code and determine the identity of the patient interface and/or its history as recorded within a storage medium on the medical instrument. This procedure can ensure that a new patient interface is used for each procedure and that the patient interface has been properly used. The use of an ID tag can also prevent the medical instrument from being operated unless the patient interface is in place. For example, the medical instrument could be programmed to stay in an idle mode unless an appropriate ID tag is present.

In an alternative embodiment, an RFID tag is used for identification and history purposes. In some embodiments, such tags may record the history of the patient interface to which they are attached. For example, each step of a medical procedure may be recorded in the RFID tag. Thus, if each step of the procedure is not performed in the proper order, the operator can be alerted and the medical instrumentation can be disabled to prevent improper use. In one embodiment, an RFSAW tag is used for identification purposes. RFSAW tags have the advantageous feature that they can withstand certain sterilization procedures (e.g., gamma irradiation) that RFID tags cannot.

When the identification and tracking tags are used on the sterile barrier as described in FIGS. 14, 15, and 16A-D, they may optionally be incorporated within the tabs 412 or 418. Because the locations of these tags are on the nonsterile surface 408 of the sheath 400, the tags do not have to be sterilized. Thus, RF ID tags, which cannot be sterilized by certain sterilization procedures, can be utilized when this sterilization requirement is limiting. For example, tab 418 may be attached after sheath 400 has been sterilized and seal 402 closed.

It will be appreciated that any suitable ID feature other than bar coding, RFID, or RFSAW may be utilized with the patient interfaces described herein.

Patient Interface for Therapeutic Ultrasound

In one embodiment, a method of use is provided wherein an acoustic couplant pad is provided, such as a gelatinous solid mass (e.g., a TPE acoustic couplant) or a gel or liquid based acoustic couplant pad, for use during an HIFU acoustic therapeutic ultrasound procedure. In one embodiment, the HIFU therapeutic ultrasound procedure is a hemostasis treatment procedure, wherein the hemostasis procedure is performed on a patient to effect bleeding cessation and closure of an access vessel following a catheterization procedure, such as after an angioplasty procedure. Typically, the couplant pad will be disposed between a patient (usually at a groin area) and a HIFU transducer configured to emit acoustic waves in order to effect bleeding cessation and coagulation at a femoral vein, artery or other vessel accessed during a catheterization procedure, thereby closing the vessel. The couplant is provided as an acoustic couplant means, as well as a thermal, microbial and sterility barrier against a therapeutic HIFU transducer. Furthermore, the acoustic couplant may have sufficient compression force transmission to allow application of sufficient force to the vessel access site with the couplant to effect temporary cessation of bleeding while the HIFU ultrasound energy is being applied. Such a procedure is described in more detail in U.S. Pat. No. 6,656,136, which is incorporated herein by reference in its entirety. In other embodiments, acoustic hemostasis is applied to effect cessation at internal bleeding sites, such as bleeding from an internal organ. In still other embodiments, the HIFU therapeutic ultrasound procedure for use with the acoustic couplant pads described herein is used for thermal ablation, such as ablation of benign or malignant tumors. Other applications of HIFU therapeutic ultrasound are well known in the art and may be used with the acoustic couplant pads disclosed herein.

Patient Interface Kits

In one embodiment, any of the components described above, including an appropriate transducer apparatus configured for a specific therapeutic and/or diagnostic purpose, lubricating liquids or means, a solid acoustic sheet or couplant device, a gel or liquid based acoustic couplant pad, a presterilized sheath, etc. are provided. In one embodiment, the methods and devices described above may be provided in one or more medical kits for use during diagnostic or therapeutic ultrasound. The kits may comprise various embodiments of the present invention and instructions for use. Optionally, such kits may further include any of the other system components described in relation to the present invention as well as any other materials or items relevant to the present invention. Preferably, such kits will be provided pre-sterilized and packaged for ease of access and use. In some embodiments, kits provide a single use, disposable patient interface. In other embodiments kits provided a patient interface that may be reused.

Other systems, methods, features and advantages of the present invention will be or become apparent to one skilled in the art upon examination of the drawings and description herein. It is intended that all additional features, advantages, etc. be included into the description of the invention, be within the scope of the invention, and be protected by the accompanying claims.

1. An ultrasound coupling pad, comprising a gelatinous thermoplastic elastomer mass, said mass adapted to permit transmission of ultrasound energy through said mass, said mass comprising a patient surface configured to transmit the acoustic energy to tissues of a patient either directly or through other materials and a transducer surface configured to receive the acoustic energy from one or more ultrasound transducers either directly or through other materials.

2. The ultrasound coupling pad of claim 1, wherein the gelatinous thermoplastic elastomer mass comprises a plurality of thermoplastic elastomers having different compositions.

3. The ultrasound coupling pad of claim 1, further comprising a means for coupling the gelatinous thermoplastic elastomer mass to an ultrasound applicator.

4. The ultrasound coupling pad of claim 1, further comprising a means for coupling the gelatinous thermoplastic elastomer mass to a patient.

5. The ultrasound coupling pad of claim 1, wherein the patient surface of the gelatinous thermoplastic elastomer mass directly contacts a patient.

6. The ultrasound coupling pad of claim 1, wherein an acoustic gel or liquid is disposed between the patient surface of the gelatinous thermoplastic elastomer mass and a patient.

7. The ultrasound coupling pad of claim 1, wherein the transducer surface of the gelatinous thermoplastic elastomer mass directly contacts an ultrasound transducer.

8. The ultrasound coupling pad of claim 1, wherein an acoustic gel or liquid is disposed between the transducer surface of the gelatinous thermoplastic elastomer mass and an ultrasound transducer.

9. The ultrasound coupling pad of claim 1, wherein the gelatinous thermoplastic elastomer mass comprises styrene.

10. The ultrasound coupling pad of claim 1, wherein the gelatinous thermoplastic elastomer mass comprises one or more soft block segments selected from the group consisting of butadiene, isoprene, isoprene-butadiene, ethylene-butylene, ethylene-propylene, ethylene-butylene-ethylene-propylene, and ethylene-ethylene-propylene.

11. The ultrasound coupling pad of claim 1, wherein the gelatinous thermoplastic elastomer mass comprises an oil.

12. The ultrasound coupling pad of claim 1, wherein the patient surface is convex shaped.

13. The ultrasound coupling pad of claim 1, further comprising a reservoir containing an acoustic gel or liquid, wherein said reservoir is adapted to dispense said acoustic gel or liquid onto said patient and/or transducer surfaces.

14. An ultrasound coupling pad, comprising:
a gelatinous thermoplastic elastomer mass, said mass adapted to permit transmission of ultrasound energy through the mass; and
a housing contacting at least some surfaces of the mass for stably holding said mass, said housing adapted to couple to an ultrasound applicator, wherein when said housing is coupled to said ultrasound applicator, at least one surface of the mass is held in close proximity to one or more ultrasound transducers in said ultrasound applicator.

15. The ultrasound coupling pad of claim 14, wherein the housing comprises a tab or tab receptacle for coupling to the ultrasound applicator.

16. The ultrasound coupling pad of claim 14, wherein the housing comprises threads for coupling to the ultrasound applicator.

17. An ultrasound coupling pad, comprising:
a gelatinous thermoplastic elastomer mass, said mass adapted to permit transmission of ultrasound energy through the mass; and
a housing contacting at least some surfaces of the mass for stably holding the mass, said housing comprising an adhesive coating on a least a portion of the housing's outer surface, said adhesive adapted to adhere the housing to a patient, wherein when said housing is adhered to said patient, at least one surface of the mass is held in close proximity to the patient.

18. The ultrasound coupling pad of claim 17, wherein the gelatinous thermoplastic elastomer mass can be removed from the housing while the housing is adhered to the patient.

19. An ultrasound coupling pad, comprising:
a first gelatinous solid mass, said first mass optimized to permit transmission of ultrasound energy having a first frequency through said mass; and
a second gelatinous solid mass, said second mass comprising a different chemical composition than the first mass and optimized to permit transmission of ultrasound energy having a second frequency through said mass, wherein said second frequency is different from said first frequency.

20. The ultrasound coupling pad of claim 19, wherein said first and second gelatinous solid masses comprise hydrogels.

21. The ultrasound coupling pad of claim 19, wherein said first and second gelatinous solid masses comprise thermoplastic elastomers.

22. The ultrasound coupling pad of claim 19, wherein said first gelatinous solid mass comprises a hydrogel and said second gelatinous solid mass comprises a thermoplastic elastomer.

23. The ultrasound coupling pad of claim 19, wherein said first gelatinous solid mass is optimized to permit transmission of ultrasound energy from an imaging ultrasound transducer and said second gelatinous solid mass is optimized to permit transmission of ultrasound energy from a therapeutic ultrasound transducer.

24. A sterile barrier for use between a patient and an instrument, comprising:
a flexible sheath adapted to prevent passage of microbes from one side of the sheath to the other;
said sheath comprising an openable seal, wherein when said seal is closed, said seal prevents passage of microbes from one side of the seal to the other;
said sheath configured to have a predeployed state and a postdeployed state, wherein in said predeployed state, the seal is closed and the flexible sheath with closed seal form a continuous barrier having no opening therein and having no edges, said continuous-barrier having an inside surface and an outside surface, wherein said inside surface is sterilized, and wherein in the postdeployed state, the flexible barrier is inverted such that the sterilized inside surface faces outward and the outside surface faces inward and is placed in contact with a medical instrument, thereby providing a barrier between the medical instrument and the sterilized surface of the sheath.

25. The sterile barrier of claim 24, wherein said flexible sheath comprises polyurethane or polyethylene.

26. The sterile barrier of claim 24, wherein at least a portion of the flexible sheath comprises material that is more rigid than other portions of the flexible sheath.

27. The sterile barrier of claim 24, wherein said seal comprises an adhesive.

28. The sterile barrier of claim 24, wherein said seal comprises Tyvek®.

29. The sterile barrier of claim 24, wherein said seal comprises a heat induced seal.

30. The sterile barrier of claim 24, further comprising one or more tabs attached to said outside surface.

31. The sterile barrier of claim 30, wherein at least one of said tabs comprises a bar code.

32. The sterile barrier of claim 30, wherein at least one of said tabs comprises an RFID feature.

33. The sterile barrier of claim 30, wherein at least one of said tabs comprises an RFSAW feature.

34. A sterile barrier for use between a patient and an ultrasound applicator, comprising:
a flexible sheath adapted to prevent passage of microbes from one side of the sheath to the other, said sheath adapted to surround an ultrasound applicator; and
a gelatinous solid mass adapted to permit transmission of ultrasound energy through said mass and prevent passage of microbes from one side of the mass to the other, said mass coupled to the flexible sheath such that when the sheath surrounds the ultrasound applicator, the mass may be placed in close proximity to one or more ultrasound transducers in the ultrasound applicator.

35. The sterile barrier of claim 34, wherein said mass is coupled to the sheath by a housing that is coupled to said flexible sheath and contacts at least some surfaces of said gelatinous solid mass and is adapted to couple said gelatinous solid mass to said ultrasound applicator.

36. An ultrasound coupling pad kit, comprising:
a sterilized gelatinous thermoplastic elastomer mass, said mass adapted to permit transmission of ultrasound energy through said mass; and
a protective barrier surrounding at least a portion of the mass, said barrier adapted to prevent passage of microbes from one side of the barrier to the other, thereby maintaining sterility of the mass, at least a portion of the barrier adapted to be removed from surrounding the mass prior to use of the mass for transmission of ultrasound energy.

37. The ultrasound coupling pad kit of claim 36, wherein said protective barrier comprises a polymer sheet.

38. The ultrasound coupling pad kit of claim 36, wherein the polymer sheet is constructed of material selected from the group consisting of polyurethane, Teflon, mylar, and polyethylene.

39. A kit for a sterile barrier for use between a patient and an ultrasound applicator, comprising:
a flexible sheath adapted to prevent passage of microbes from one side of the sheath to the other, said sheath adapted to surround an ultrasound applicator, at least one surface of the flexible sheath sterilized; and
a gelatinous solid mass adapted to permit transmission of ultrasound energy through said mass, said mass comprising a sterilized patient surface configured to transmit the acoustic energy to tissues of a patient either directly or through other materials and a transducer surface configured to receive the acoustic energy from one or more ultrasound transducers in said ultrasound applicator either directly or through other materials.

40. The kit of claim 39, wherein said gelatinous solid mass is coupled to the flexible sheath.

41. An ultrasound coupling pad, comprising:
a gelatinous thermoplastic elastomer mass, said mass adapted to permit transmission of ultrasound energy through said mass; and
a means for coupling said mass to an ultrasound applicator.

42. An ultrasound coupling pad, comprising:
a gelatinous thermoplastic elastomer mass, said mass adapted to permit transmission of ultrasound energy through said mass; and
a means for coupling said mass to a patient.

43. A sterile barrier for use between a patient and an ultrasound applicator, comprising:
a gelatinous solid mass, said mass adapted to permit transmission of ultrasound energy through said mass and prevent passage of microbes from one side of the mass to the other;
a means for preventing passage of microbes from at a least a portion of an ultrasound applicator's surface to a patient;
a means for coupling said gelatinous solid mass to said means for preventing passage of microbes.

44. A method of transmitting ultrasound energy from an ultrasound transducer to tissue of a patient, comprising:
positioning one surface of a gelatinous thermoplastic elastomer mass in close proximity to an ultrasound transducer, said mass adapted to permit transmission of ultrasound energy through said mass;
positioning another surface of the gelatinous thermoplastic elastomer mass in close proximity to tissue of a patient; and
energizing the ultrasound transducer such that ultrasound energy passes from the ultrasound transducer, through the mass, and into the tissue of the patient.

45. The method of claim 44, wherein said step of positioning a surface of the mass in close proximity to an ultrasound transducer comprises directly contacting the ultrasound transducer with the mass.

46. The method of claim 44, wherein said step of positioning a surface of the mass in close proximity to tissue of a patient comprises directly contacting the tissue with the mass.

47. The method of claim 44, wherein said step of positioning a surface of the mass in close proximity to an ultrasound transducer comprises applying an acoustic gel or liquid between the mass and the ultrasound transducer.

48. The method of claim 44, wherein said step of positioning a surface of the mass in close proximity to tissue of a patient comprises applying an acoustic gel or liquid between the mass and the patient.

49. The method of claim 44, wherein said step of positioning a surface of the mass in close proximity to an ultrasound transducer comprises coupling the mass to the ultrasound transducer.

50. The method of claim 44, wherein said step of positioning a surface of the mass in close proximity to tissue of a patient comprises coupling the mass to the patient.

51. The method of claim 44, wherein said step of positioning a surface of the mass in close proximity to an ultrasound transducer is performed prior to said step of positioning a surface of the mass in close proximity to tissue of a patient.

52. The method of claim 44, wherein said step of positioning a surface of the mass in close proximity to tissue of a patient is performed prior to said step of positioning a surface of the mass in close proximity to an ultrasound transducer.

53. A method of acoustic hemostasis, comprising:
positioning one surface of a gelatinous solid mass in close proximity to a wound on a patient, said mass adapted to permit transmission of ultrasound energy through said mass;
applying sufficient pressure to the gelatinous solid mass so as to temporarily stop or slow bleeding from said wound; and
transmitting ultrasound energy through said mass into said wound, thereby stopping bleeding from said wound.

54. The method of claim 53, wherein said step of applying pressure to the mass comprises contacting the mass with an ultrasound applicator and applying force to the ultrasound applicator.

55. A method of providing a sterile barrier between a patient and a medical instrument, comprising:
opening a seal in a flexible sheath, said sheath adapted to prevent passage of microbes from one side of the sheath to the other, the seal disposed on said sheath, wherein when said seal is closed, said seal prevents passage of microbes from one side of the seal to the other, wherein prior to opening the seal, the flexible sheath with closed seal forms a continuous barrier having no opening therein and having no edges, said continuous barrier having an inside surface and an outside surface, wherein said inside surface is sterilized;
contacting a medical instrument with said outside surface of the flexible sheath; and
inverting the flexible sheath so that the sterilized inside surface faces outward and the outside surface faces inward in contact with the medical instrument, thereby providing a barrier between the medical instrument and the sterilized surface of the sheath.

56. The method of claim 55, wherein said opening of the seal and inverting of the flexible sheath is accomplished by pulling on tabs fixed on said outside surface.

57. The method of claim 55, wherein said step of opening of the seal is performed prior to said step of contacting a medical instrument with said outside surface.

58. The method of claim 55, wherein said step of contacting a medical instrument with said outside surface is performed prior to said step of opening of the seal.

59. The method of claim 55, wherein said opening, contacting, and inverting steps are performed by non-sterile personnel.

60. The method of claim 55, wherein said contacting step is performed by partially inverting said flexible sheath prior to said opening step.

61. A method of providing a sterile barrier for use between a patient and an ultrasound applicator, comprising:
inserting an ultrasound applicator within a flexible sheath adapted to prevent passage of microbes from one side of the sheath to the other; and
coupling a gelatinous solid mass to the ultrasound applicator in close proximity to one or more ultrasound transducers, the gelatinous solid mass adapted to permit transmission of ultrasound energy through said mass and prevent passage of microbes from one side of the mass to the other.

62. The method of claim 61, wherein said gelatinous solid mass is coupled to said flexible sheath such that the coupling step is performed after the ultrasound applicator is at least partially inserted within the flexible sheath.

63. The method of claim 61, wherein the coupling step comprises sandwiching the flexible sheath between the ultrasound applicator and the gelatinous solid mass.

64. A method of providing a sterile barrier for use between a patient and an ultrasound applicator, comprising:
coupling a gelatinous solid mass to an ultrasound applicator in close proximity to one or more ultrasound transducers, the gelatinous solid mass adapted to permit transmission of ultrasound energy through said mass and prevent passage of microbes from one side of the mass to the other, said mass comprising a sterilized patient surface and a protective barrier applied to the surface, whereby sterility of the surface is maintained; and
removing the protective barrier from the patient surface.

65. The method of claim 64, wherein said removing step is performed after said coupling step.

66. The method of claim 64, wherein said protective barrier comprises a film and said removing step comprises peeling off the film from the patient surface.

67. A method of providing a sterile barrier for use between a patient and an ultrasound applicator, comprising:
removing a protective barrier from a patient surface of gelatinous solid mass, the mass adapted to permit transmission of ultrasound energy therethrough and prevent passage of microbes from one side of the mass to the other, the protective barrier applied to the patient surface of the mass to maintain sterility of the surface prior to use; and
coupling the gelatinous solid mass to a patient so that the patient surface is in close proximity to the patient.

68. A method of optimizing a thermoplastic elastomer for use as an acoustic coupler, the thermoplastic elastomer comprising a soft block segment, a hard block segment, and one or more modifier, the modifier selected from the group consisting of a hard block compatible modifier and a soft block compatible modifier, the method comprising varying at least one of the soft block segment, the hard block segment, and the modifier until an elastomer having one or more desired properties is obtained.

69. The method of claim 68, wherein said varying comprises varying the relative amounts of the soft block segment, hard block segment, and the modifier.

70. The method of claim 68, wherein said varying comprises varying the composition of at least one of the soft block segment, the hard block segment, and the modifier.

71. The method of claim 68, wherein at least one desired property is an acoustic property.

72. The method of claim 71, wherein the acoustic property comprises acoustic impedance.

73. The method of claim 71, wherein the acoustic property comprises acoustic attenuation.

74. The method of claim 68, wherein at least one desired property is a mechanical property.

75. The method of claim 74, wherein the mechanical property comprises compression force transmission.

76. The method of claim 74, wherein the mechanical property comprises elasticity.

77. The method of claim 68, wherein at least one desired property is a thermal insulative property.
